(19) United States
(12) Patent Application Publication
(10) Pub. No.: US 2023/0172810 A1
(43) Pub. Date: Jun. 8, 2023

(54) MILK STORAGE SYSTEM

(71) Applicant: Lansinoh Laboratories, Inc., Alexandria, VA (US)

(72) Inventors: Peter Visconti, Chicago, IL (US); Yuka Yamaguchi, Alexandria, VA (US); Selim Tekin, Izmir (TR); Simone Stadtmueller, Berlin (DE); Luke A. Westhoff, Kenosha, WI (US); Alec Devlin, Racine, WI (US); Patrick Tetzlaff, Caledonia, WI (US); Brian T. Leadingham, Pleasant Prairie, WI (US); Alex J. Gruber, Hubertus, WI (US)

(21) Appl. No.: 18/061,230

(22) Filed: Dec. 2, 2022

Related U.S. Application Data
(60) Provisional application No. 63/266,913, filed on Jan. 19, 2022.
(60) Provisional application No. 63/264,916, filed on Dec. 3, 2021.

(51) Int. Cl. A61J 9/08; B65D 21/02

(52) U.S. Cl. CPC A61J 9/085; B65D 21/0231

(57) ABSTRACT

A system for collecting, storing, and dispensing breastmilk can include stackable containers with various shaped lids and bottles that are shaped complementary to each other to provide stability to the stacked container assembly. In some examples, the lids and bottle also include attachment features that engage with each other to secure a bottle of a top container to the lid of a bottom container.

### RELATED APPLICATION(S)

This patent application is related to U.S. Patent Application No. 63/266,913 filed on Jan. 19, 2022, the entirety of which is hereby incorporated by reference. This patent application is also related to U.S. Patent Application No. 63/264,916 filed on Dec. 3, 2021, the entirety of which is hereby incorporated by reference.

### BACKGROUND

Breastmilk contains all the nutrients a baby needs for the first six months of life to support growth and development. Therefore, breastfeeding and/or breastmilk feeding is recommended globally by the World Health Organization and healthcare professionals. When possible, mothers should be supported to initiate breastfeeding within the first hour of their baby's life, breastfeed exclusively for six months, and continue breastfeeding while giving appropriate complementary foods for up to two years of age or beyond. There are situations, however, when direct breastfeeding is not possible. For example, babies may not be able to latch and suckle, due to cleft palate or tongue tie, prematurity, low birthweight, or other causes, and mothers may not be able to breastfeed, due to medical conditions or recovery from birth trauma. In such cases, human breastmilk feeding becomes the next best option. Capturing breastmilk is beneficial for mothers who want to provide their infants with natural breastmilk. For many mothers, a breast pump plays a significant role in the breastmilk feeding process. Breast pumps can also be essential tools for mothers to capture milk for later use, which can be especially useful for mothers that are traveling, working, or otherwise away from their infants. Pumping is also useful to relieve engorgement and milk build up in the breast.

The principle methods used by mothers to collect human breastmilk are hand expressing breastmilk into a container and expressing breastmilk into a container using a manual or electric breast pump. In some situations, after expressing the breastmilk into a collection container, the breastmilk is not immediately consumed by a baby. In such cases, it may be desirable to store or transport the breastmilk while it is within the collection container. If the mother is able to pump more breastmilk than the baby can consume in a given period, the situation may arise where the mother is left with multiple containers of breastmilk to store or transport.

### SUMMARY

Regularly expressing breastmilk, whether by nursing or pumping, is a critical factor for mothers to establish and maintain breastmilk supply, even if an infant is not nursing at all or not nursing well. This disclosure describes system that offers flexibility and convenience for pumping, collecting, and storing expressed breastmilk. In some examples, mothers may express more breastmilk than may be consumed by a baby in a given time period. In such cases, it may be desirable to store the breastmilk. When collected in a container, the mother may wish to conveniently store the breastmilk in that container, rather than transfer it into a separate container for storage. Likewise, when providing the expressed breastmilk to a baby, the mother may wish to conveniently provide the breastmilk to the baby from that container, rather than transfer the breastmilk from a storage container to a consuming container for a baby. One aspect of this invention includes a container that may be used for collecting breastmilk, storing breastmilk, and providing breastmilk to a baby without the need to transfer the breastmilk into different containers.

In general terms, this disclosure is directed to breastmilk storage systems. In one possible configuration and by non-limiting example, the breastmilk storage system includes containers that are configured to stack on top of each other to save shelf space in a refrigerator. The bottle and lids of the containers include matching geometry to provide stability to the container stacks and prevent the top containers from slipping of the bottom containers. In some examples, the lids and bottles of the containers include attachment features, such as interference fitting protruding rings and grooves, or threads, to help secure the top containers to the bottom containers and prevent them from being knocked off of the bottom containers. Various aspects are described in this disclosure, which include, but are not limited to, the following aspects.

One aspect is a breastmilk storage system. The breastmilk storage system comprises a first bottle with a first associated lid and a second bottle. The second bottle is stacked on top of the first associated lid while the first associated lid is covering an opening of the first bottle.

Another aspect is a method for stacking breastmilk storage containers. The method comprises providing a first bottle with a first associated lid and a second bottle. The method further comprises positioning the second bottle on top of the first associated lid while the first associated lid is covering an opening of the first bottle.

### BRIEF DESCRIPTION OF THE DRAWINGS

FIG. 1 is a perspective view of an example of a breastmilk collection system.

FIG. 2 is a front view of an example breastmilk storage system.

FIG. 3 is a front view of an example breastmilk container of the breastmilk storage system of FIG. 2.

FIG. 4 is a front view of an example lid of the example breastmilk container of FIG. 3.

FIG. 5 is a cross-sectional side view of an example bottle of the breastmilk container of FIG. 3.

FIG. 6 is a cross-sectional view of a portion of another example breastmilk storage system.

FIG. 7 is a front view of a portion of another example passive attachment breastmilk storage system.

FIG. 8 is a front view of a portion of another example passive attachment breastmilk storage system.

FIG. 9 is a schematic view of the example passive attachment breastmilk storage system of FIG. 7.

FIG. 10 is a front view of another example container that can be used with a passive attachment breastmilk storage system.

FIG. 11 is a front view of another example passive attachment breastmilk storage system.

FIG. 12 is a front view of another example passive attachment breastmilk storage system.

FIG. 13 is a front view of another example passive attachment breastmilk storage system.

FIG. 14 is a cross sectional view of another example passive attachment breastmilk storage system.

FIG. 15 is a perspective view of an example container of another example passive attachment breastmilk storage system.

FIG. 16 is a front view of the example container of FIG. 15.

FIG. 17 is a cross-sectional view of the example container of FIG. 15.

FIG. 18 is another cross-sectional view of the example container of FIG. 15.

FIG. 19 is a perspective view of another example passive attachment breastmilk storage system using the container of FIG. 15.

FIG. 20 is a front view of the example breastmilk storage system of FIG. 19.

FIG. 21 is a cross-sectional view of the example breastmilk storage system of FIG. 19.

FIG. 22 is an alternative cross-sectional view of the example breastmilk storage system of FIG. 19.

FIG. 23 is a semi-transparent front view of a portion of another example passive attachment breastmilk storage system.

FIG. 24 is semi-transparent front view of a portion of another example passive attachment breastmilk storage system.

FIG. 25 is a cross-sectional view of another example passive attachment breastmilk storage system.

FIG. 26 is a front view of another example container.

FIG. 27 is a front view of another example breastmilk storage system using the container of FIG. 26.

FIG. 28 is a front view of another example breastmilk storage system.

FIG. 29 is a front view of another example breastmilk storage system.

FIG. 30 is a front view of another example container.

FIG. 31 is a front view of a portion of another example breastmilk storage system using the container of FIG. 30.

FIG. 32 is a cross-sectional view of a portion of another example breastmilk storage system.

FIG. 33 is a cross-sectional view of an example bottle.

FIG. 34 is a cross-sectional view of the example bottle of FIG. 33, further including directional arrows.

FIG. 35 is a cross-sectional view of another example bottle.

FIG. 36 is a front view of another example passive attachment breastmilk storage system without the lid.

FIG. 37 is a cross-sectional view of an example active attachment breastmilk storage system.

FIG. 38 is a cross-sectional view of a container of the active attachment breastmilk storage system of FIG. 37.

FIG. 39 is another cross-sectional view of a container of the active attachment breastmilk storage system of FIG. 37.

FIG. 40 is a front view of the container of the active attachment breastmilk storage system of FIG. 37.

FIG. 41 is a cross-sectional view of the active attachment breastmilk storage system of FIG. 42.

FIG. 42 is a cross-sectional view of a portion of the active attachment breastmilk storage system of FIG. 37.

FIG. 43 is a front view of the active attachment breastmilk storage system of FIGS. 37.

FIG. 44 is a cross-sectional view of another active attachment breastmilk storage system.

FIG. 45 is a front view of an example container with a nipple attachment used in place of a lid.

FIG. 46 is a front view of the example container of FIG. 45 with the nipple attachment shown in an alternate configuration.

FIG. 47 is a front view of the example container of FIG. 45 including an example cover.

FIG. 48 is a front view of the example container of FIG. 45 including another example cover.

FIG. 49 is a front view of the example container of FIG. 45 including another example cover.

FIG. 50 is a front view of the example container of FIG. 45 including another example cover.

FIG. 51 is a front view of another example container including a nipple attachment.

FIG. 52 is a front view of another example container with a breast contacting attachment used in place of a lid.

FIG. 53 is a front view of another example container with a penetrable lid.

FIG. 54 is front view of another example container.

FIG. 55 is front view of another example passive attachment breastmilk storage system using the example container of FIG. 54.

FIG. 56 is a side view of another example container.

FIG. 57 is a front view of another example passive attachment breastmilk storage system using the example container of FIG. 56.

FIG. 58 is a bottom perspective view of another example bottle.

FIG. 59 is a top perspective view of another example lid.

FIG. 60 is a semi-transparent to perspective view of the example bottle of FIG. 58 stacked on top of the example lid of FIG. 59.

FIG. 61 is a front view of the example bottle of FIG. 58 stacked on top of the example lid of FIG. 59.

FIG. 62 is a front view of another example container.

FIG. 63 is a front view of a hybrid attachment breastmilk storage system using the example container of FIG. 62.

FIG. 64 is another front view of the hybrid attachment breastmilk storage system of FIG. 63.

FIG. 65 is a front perspective view of another example container.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

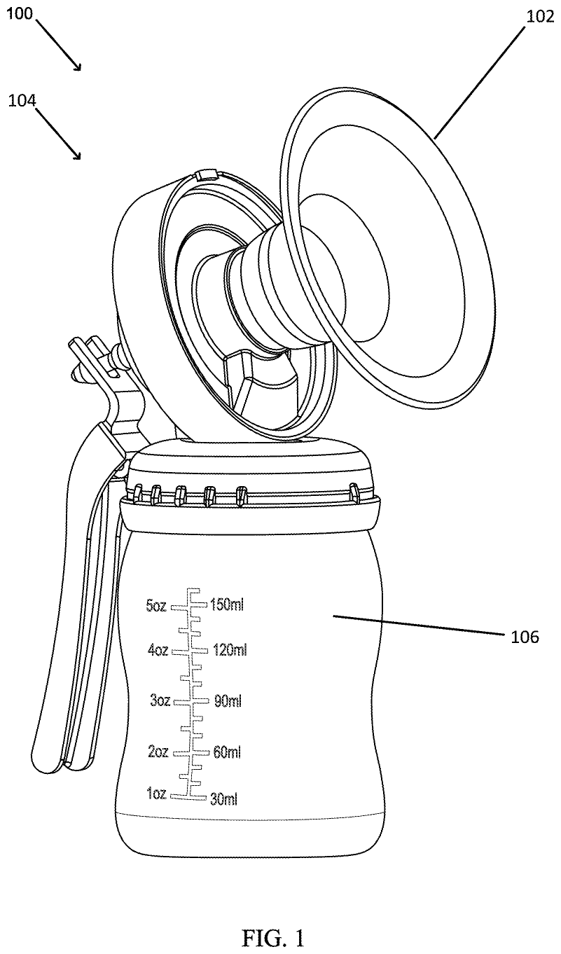

The disclosed technology relates to breast pumps. Examples disclosed herein can help breast pump users efficiently express and store milk. An example breast pump system that can operate to collect and store breast milk is described in FIG. 1.

FIG. 1 is a perspective view of an example of a breastmilk collection system 100. In this embodiment, the breastmilk collection system 100 is handheld and uses a pump. In this embodiment, the breastmilk collection system 100 uses a manual pumping mechanism rather than a motorized pump, although the collection system is able to operate using both manual and motorized pumps. The breastmilk collection system 100 includes a breast contacting flange 102, a pump 104, and a bottle 106.

The breastmilk collection system operates to pump milk from a woman's breast and collect the milk in the bottle. The breast contacting flange 102 of the breastmilk collection system 100 is placed on woman's breast, at which point, the pump is operated to pump the breastmilk from the breast. After the breastmilk is expressed from the breast, it is collected in the bottle 106.

In some embodiments, the bottle 106 is detachable from the pump 104 and breast contacting flange 102. Detaching the milk bottle allows the bottle containing the breastmilk to be used to be used as a hand expression capture container or a system to feed a baby, or store or transport the breastmilk. Detaching the bottle from the other parts of the breastmilk collection system also allows the component parts of the breastmilk collection system to be more easily cleaned. In some embodiments, multiple bottles can be used with the breastmilk collection system so that as one bottle is filled, it can be detached from the breastmilk collection system and another bottle can be attached in its place.

In some cases, the woman may pump more milk than the baby is able to consume in a given time period. In this case, the breastmilk may need to be stored or transported in the milk bottles.

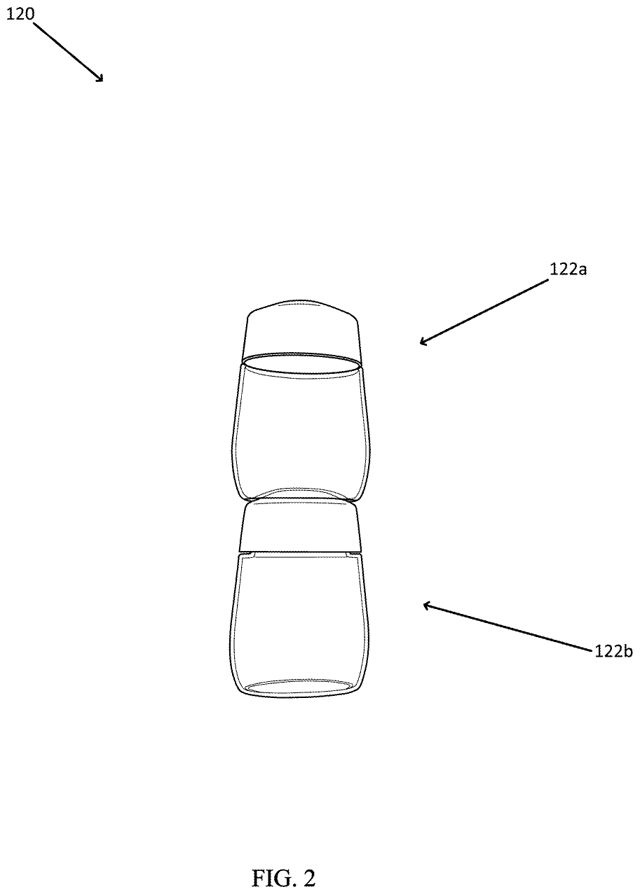

FIG. 2 is a front view of an example breastmilk storage system 120. The breastmilk storage system includes two containers, consisting of a top container 122a and a bottom container 122b, stacked vertically on top of each other. Although two containers are used in the example of FIG. 2, in other embodiments, the breastmilk storage system 120 includes more than two containers stacked on top of each other. Utilizing the breastmilk storage system 120, breastmilk containers 122a, 122b can be stored or transported more efficiently so that more breastmilk containers can be stored utilizing less shelving space.

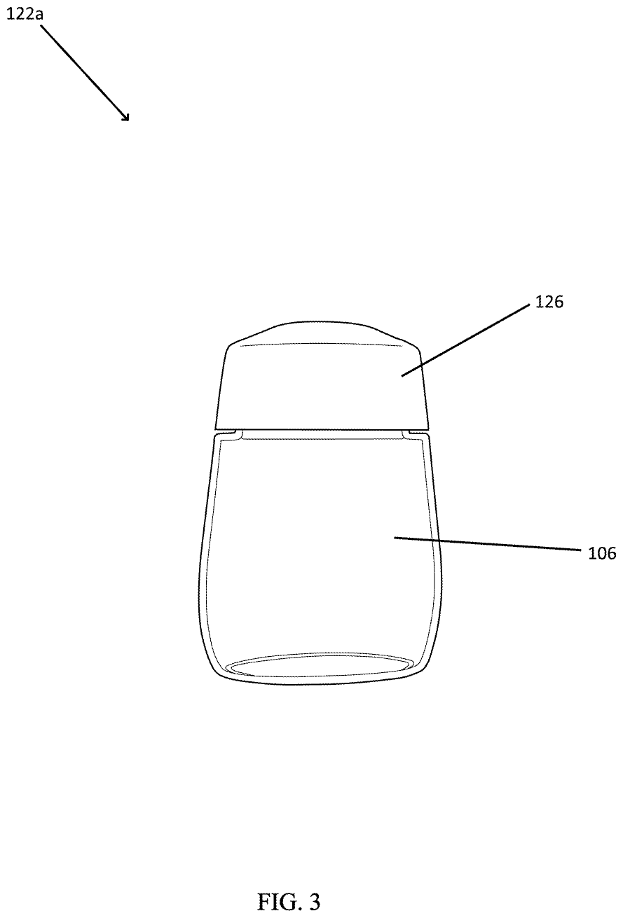

FIG. 3 is a front view of a breastmilk container 122a of the breastmilk storage system 120 of FIG. 2. The breastmilk container 122a includes a bottle 106 and a lid 126. The lid 126 of the breastmilk container 122a is placed on top of the bottle 106 to seal off an interior of the bottle 106 so that its contents (i.e., breastmilk) are contained therein. Thus, breastmilk can be securely stored in the container 122a without spilling, even if the container 122a were to be knocked over or held upside down.

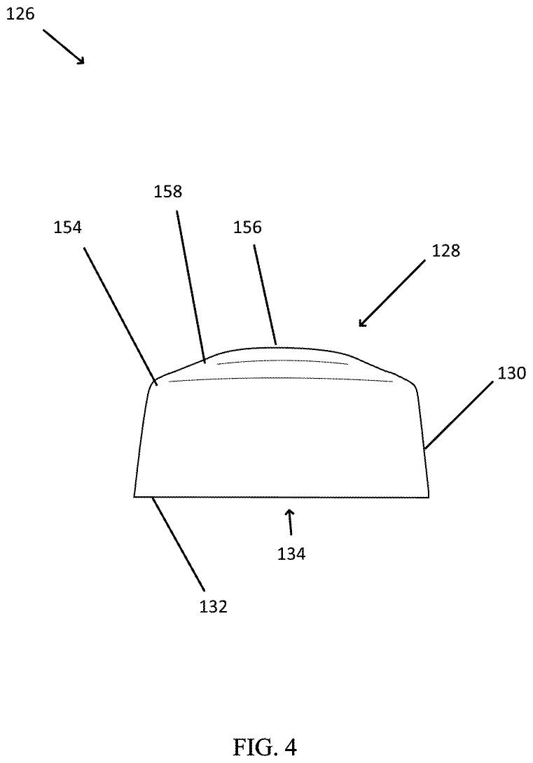

FIG. 4 is a front view of the example lid 126 of the breastmilk container 122a of FIG. 3. As seen in FIG. 4, the example lid 126 includes an upper surface 128, a side surface 130, a bottom surface 132, and an interior 134. In general, the lid 126 functions to seal an opening in the bottle 106 of the container 122a to prevent the breastmilk from exiting the bottle 106. In some examples, the lid 126 is manufactured from a ridged plastic material, however, in other examples, the lid 126 is manufactured from an elastomeric material. In some examples, the elastomeric material provides enhanced stability to the breastmilk storage system due to the non-skid properties of the elastomeric material.

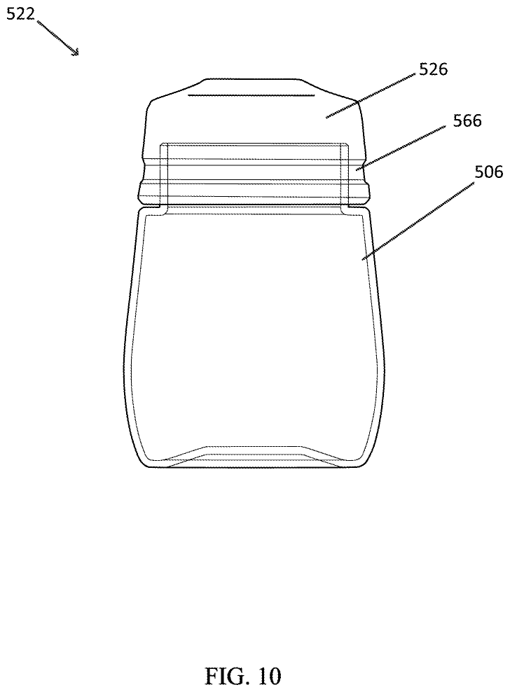

In some embodiments, as depicted in FIG. 4, the upper surface 128 of the lid 126 is rounded and dome-shaped, however, in other embodiments, the shape of the upper surface 128 may vary. In the embodiment of FIG. 4, the outline of the lid 126 is substantially circular when viewed from a top direction, however, in other embodiments, the outline of the lid 126 may take different shapes, such as an oval shape, a square shape, or some other geometrical shape. The domed shaped upper surface 128 includes an edge 154, a dome portion 156, and a slanted side 158. In the example of FIG. 10, the dome-shaped upper surface 128 includes a smooth transition between the slanted side 158 and the dome portion 156.

The side surface 130 of the lid 126 extends down from the upper surface 128 to the bottom surface 132. As seen in FIG. 4, the side surface 130 is tapered so that the circular cross section of the lid 126 is larger in diameter closer to the bottom surface 132 of the lid 126, however, in other embodiments, the side surface 130 of the lid 126 may extend vertically downwards so that the circular cross section of the lid 126 remains substantially consistent at any point along the side surface 130. In different embodiments, the side surface 130 may be tapered at higher or lower angles. Although the side surface 130 of the embodiment in FIG. 4 is depicted as being smooth, in other embodiments, the side surface 130 includes texturing or other extruded features as to facilitate the removal of the lid 126 by a user.

The bottom surface 132 of the lid 126 extends between the side surface 130 and the interior 134 of the lid 126. As depicted in FIG. 4, the side surface 130 is flat, however, in other embodiments, the side surface 130 may be a rounded edge. In some examples, when the lid 126 is removed from the bottle 106, the lid 126 may be stored on its bottom surface 132.

Generally, the interior 134 of the lid is visible when viewing the lid 126 from its bottom surface 132. In the example of FIG. 4, the lid 126 is viewed from the side surface so that the interior 134 of the lid 126 is not visible. The interior 134 extends from the bottom surface 132 of the lid 126 and into the body of the lid 126 towards the upper surface 128. The interior 134 of the lid 126 generally does not breach the upper surface 128 or side surface 130 of the lid 126, however, in some examples, the side surface 130 or upper surface 128 of the lid 126 include perforations or other openings that allow for airflow between the interior and exterior space of the container 122.

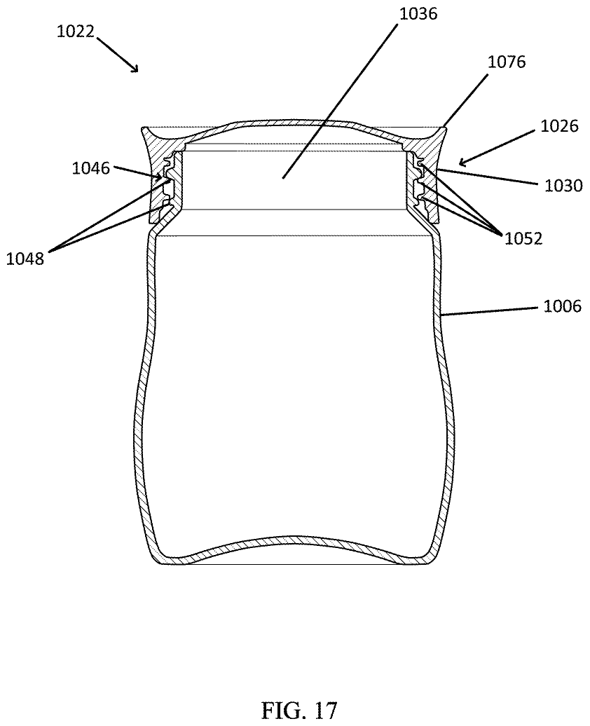

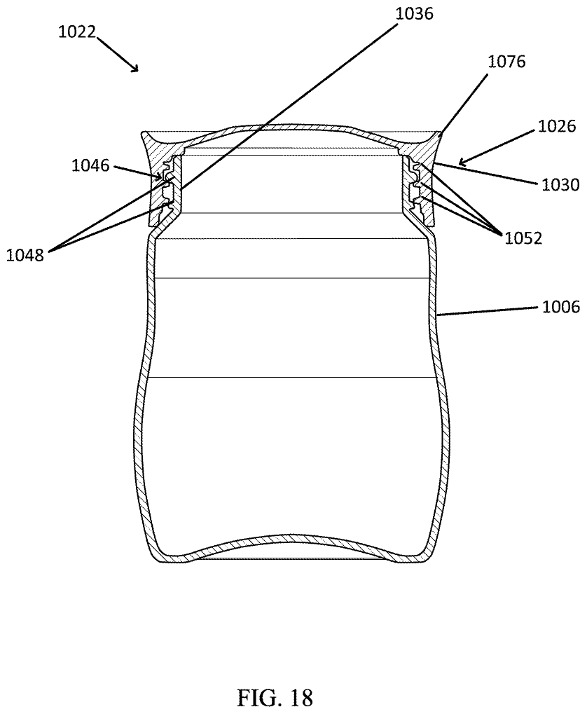

In some examples, the interior 134 of the lid has a circular cross section and includes smooth sides that extend vertically upward. In other examples, as explained with reference to FIGS. 17-18, the sides of the lid interior 134 include features for securing the lid 126 to the bottle 106.

The lid 126 may come in various sizes. In some examples, the outer diameter of the lid 126, as measured at the bottom edge of the side surface 130, is approximately 2 to 8 inches. In some examples, the overall height of the lid, as measured from the bottom-most point of the bottom surface 132 to the top of the upper surface 128 is between 1 and 6 inches.

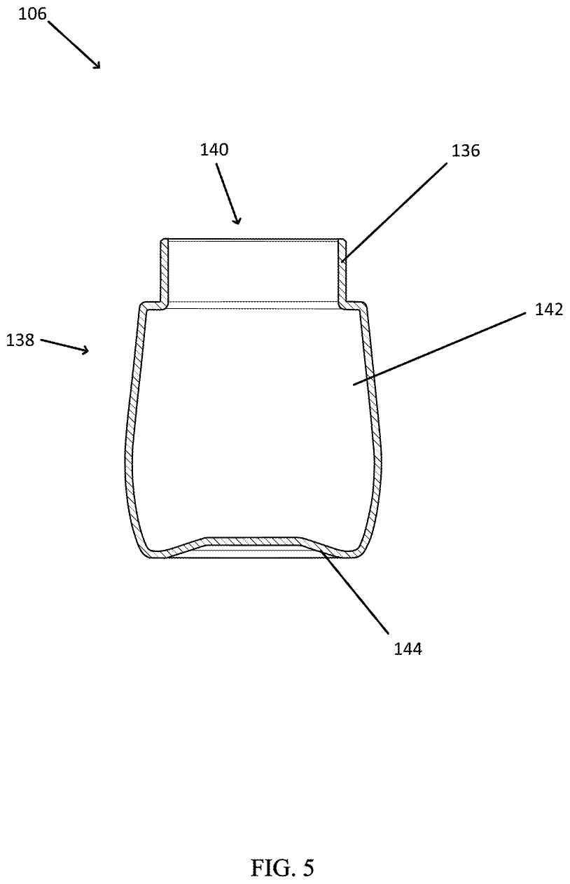

FIG. 5 is a cross-sectional side view of the example bottle 106 of the container 122a of FIG. 3. As seen in FIG. 5, the example bottle 106 includes a neck 136, a body 138, and an interior 140. In general, the bottle 106 functions to hold a volume of breastmilk in its interior 140. As explained herein, the bottle 106 may be used with a variety of different attachments, such as, for example, a pump, a lid, or a nipple.

In some examples, as depicted in FIG. 5, the neck 136 of the bottle 106 is shaped complementary to the interior 134 of the lid 126. Thus, in the example of FIG. 5, the neck 136 of the bottle 106 includes a circular cross section with a diameter similar to that of the cross-sectional diameter of the interior 134 of the lid 126. The neck 136 extends from a top of the bottle 106 down to the body 138 of the bottle. In some examples, as seen in the example of FIG. 5, the neck 136 includes a smooth, vertical outer surface. In other examples, as explained with reference to FIGS. 17-18, the neck 136 includes features that help to secure the lid 126 to the bottle 106.

The body 138 of the bottle extends from the neck 136 to the bottom of the bottle 106. The body of the bottle includes a side surface 142 and bottom surface 144 of the bottle 106. In some examples, such as the example of FIG. 5, the side surface 142 of the bottle includes a chamfered transition to the neck 136. In other examples, the side surface 142 of the bottle smoothly graduates into the neck 136 or cuts off abruptly, creating a hard edge before transitioning into the neck 136.

As seen in FIG. 5, the side surface 142 of the bottle 106 may include a wavy, ergonomic shape to facilitate easy grasping of the bottle 106 by a user, however, in other examples, the side surface 142 of the bottle 106 may be substantially cylindrical, so that the cross section of the bottle 106 remains relatively constant from the top of the bottle 106 to the bottom of the bottle 106. In some examples such as the example of FIG. 5, the body 138 of the bottle 106 is opaque, however, in other examples, the body 138 of the bottle 106 is transparent, so that the volume of breastmilk contained in the interior 140 of the bottle 106 is visible.

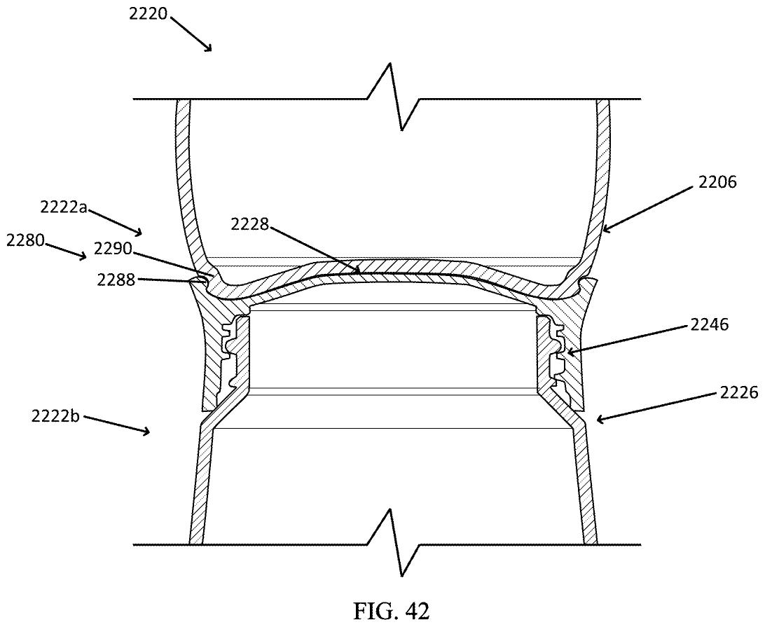

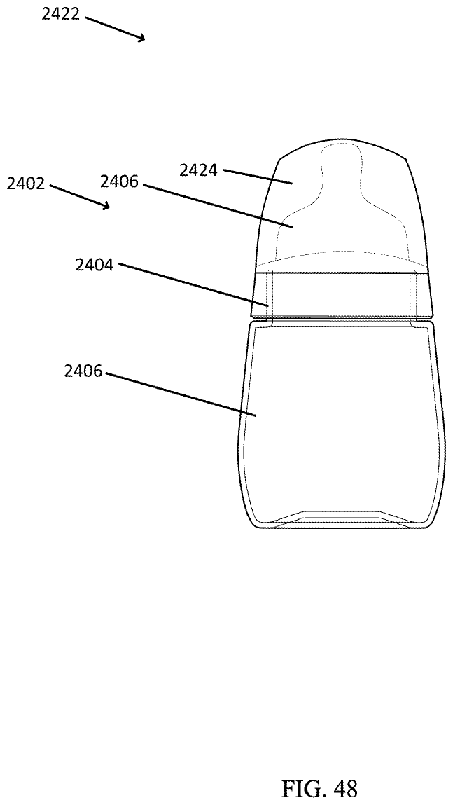

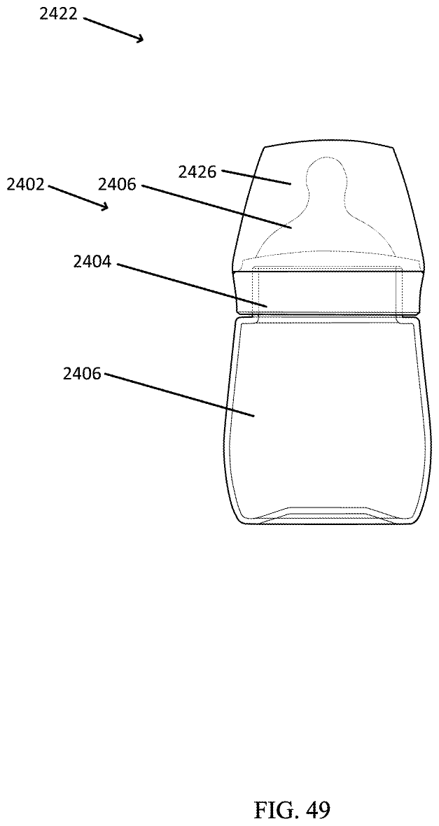

The side surface 142 of the bottle 106 may also be marked with graduations, as to help a user gauge the volume of breastmilk contained within the bottle 106. In some examples, the body 138 is smooth, however in other examples, as explained with reference to FIGS. 42-49, the body 138 may include attachment features, such as ridges, located around the side surface 142 near the bottom of the bottle 106.

The bottom surface 144 of the bottle 106 is located on the bottom side of the bottle 106. In some examples, the bottom surface 144 is flat, however, as seen in example of FIG. 5, the bottom surface 144 may also be curved. In the example of FIG. 5, the bottom surface 144 is concave inward towards the top of the bottle 106. In other examples, the bottom surface 144 includes more complex geometry, such as multiple recesses or protrusions. In the example of FIG. 5, the outer edge of the bottom surface connects to the side surface 142 of the bottle 106, and the bottom surface 144 gradually recesses upward towards the top of the bottle 106. In some examples, the geometry of the bottom surface 144 of the bottle 106 is complementary to the geometry of the upper surface 128 of the lid 126.

The interior 140 of the bottle 106 is visible when viewing the bottle 106 from the top of the bottle 106. In some examples, the interior 140 extends from the top of the bottle 106 into the body 138 of the bottle 106 towards the bottom surface 144. The interior 140 of the bottle 106 generally does not breach the side surface 142 of the body 138 or bottom surface 144 of the bottle 106. In some examples, the interior 140 of the bottle 106 has a circular cross section and smooth sides that extend vertically downwards. In other examples, the interior 140 of the bottle 106 is shaped complementary to the side surface 142 of the bottle 106 so that the material forming the sides of the bottle 106 has a relatively consistent thickness at each point on the bottle 106. The interior 140 of the bottle 106 contains the breastmilk placed into the bottle 106. In some examples, the interior 140 of the bottle 106 is sufficiently large as to hold between 10 ml and 1000 ml of breastmilk. In other examples, the interior 140 of the bottle 106 holds approximately 150 ml of breastmilk.

The bottle 106 may be made from various materials such as, for example, glass, plastic, metal, or other materials. Likewise, the bottle 106 may be made in various sizes. For example, in some embodiments, the bottle 106 is between approximately 3 and 12 inches tall, as measured from the bottommost point of the bottle 106 to the topmost point of the bottle 106. The bottle 106 may take a variety of shapes. In some examples, such as the example of FIG. 5, the cross-sectional shape of the body 138 is circular and the cross-sectional diameter of the body 138 is between 2 and 10 inches.

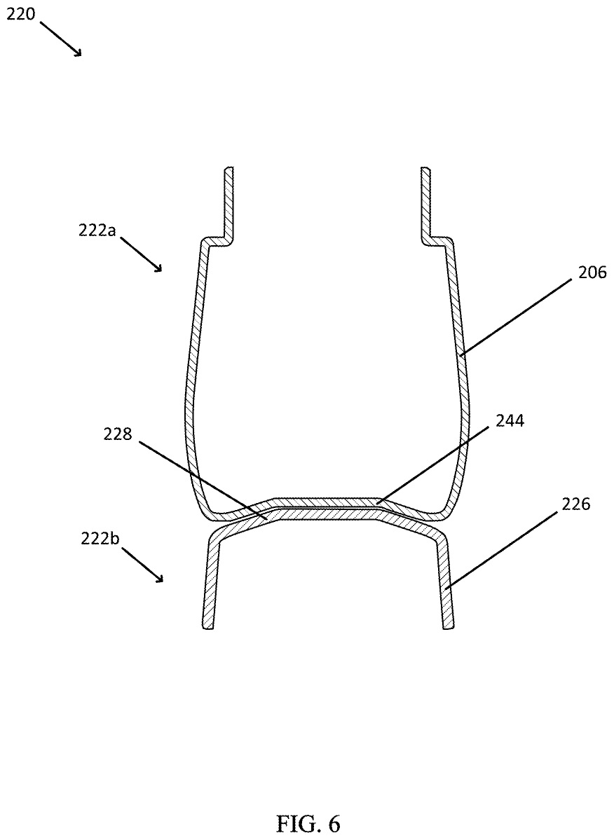

FIG. 6 is a cross-sectional view of a portion of another example breastmilk storage system 220, depicting an example passive attachment between the bottle 206 of the top container 222a and a lid 226 of the bottom container 222b. As depicted in FIG. 6, the bottom surface 244 of the bottle 206 is placed on top of the upper surface of the lid 226. The bottom surface 244 of the bottle 206 and the upper surface 228 of the lid 226 are shaped complementary to each other. The matching geometry of the lid 226 and the bottle 206 function to secure the bottle 206 resting on top of the lid 226 in place. In the example of FIG. 6, because the outer edges of the bottom surface 244 of the bottle 206 extend below the uppermost point in the center of the bottom surface 244 of the bottle 206, the outer edges provide stability to the bottle 206 resting on top of the lid 226. This configuration helps to make it more difficult for the top container 222a to fall off of the bottom container 222b.

In the example of FIG. 6, the bottom surface 244 of the bottle 206 and the upper surface 228 of the lid 226 each include three distinct sections: a horizontal section, and two angled sections that extend to the outer edges of the lid 226 and bottle 206. In other examples, however, the upper surface 228 of the lid 226 and the bottom surface 244 of the bottle 206 transition seamlessly so that there are no angles between distinguishable sections of the surfaces.

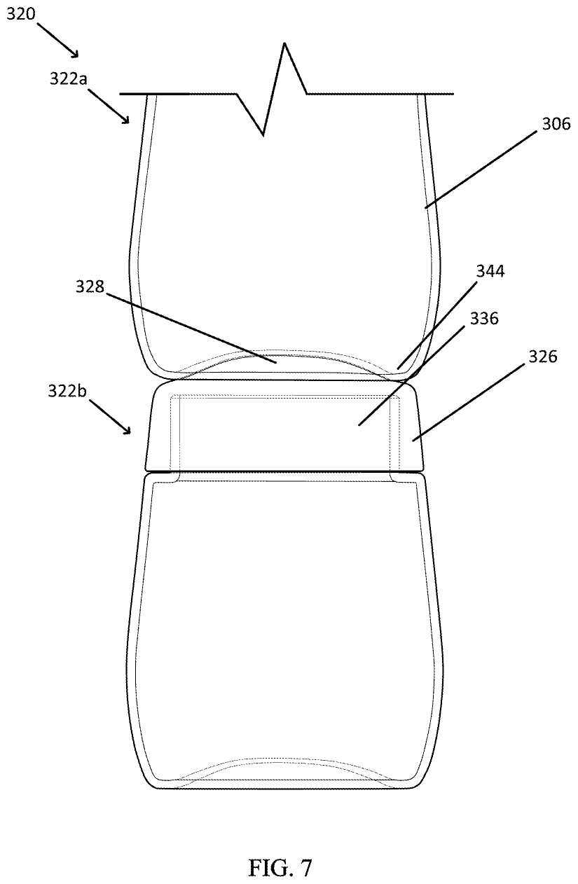

FIG. 7 is a front view of a portion of another example passive attachment breastmilk storage system 320. In the example of FIG. 7, the bottom surface 344 of a bottle 306 of a top container 322a is placed onto the upper surface 328 of a lid 326 of a bottom container 322b. In this example, the bottom container 322b also includes a bottle 306 shaped identically to the bottle 306 of the top container 322a, however, in other examples, the bottle 306 of the bottom container 322b may be shaped differently from the bottle 306 of the bottom container 322b. In this example, the bottom surface 344 of the bottle 306 of the top container 322a is flush with the upper surface 328 of the lid 326 of the bottom container 322b. The two complementary shaped surfaces mate with each other to provide stability to the breastmilk storage system 320.

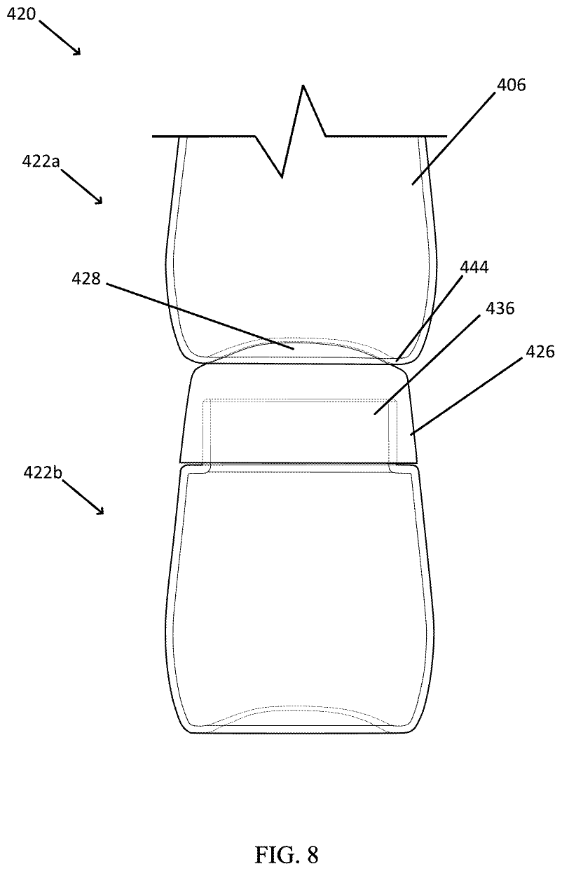

FIG. 8 is a front view of another example passive attachment breastmilk storage system 420. In this example, the lid 426 of the bottom container 422b is shaped slightly differently than the lid 326 of bottom container 322b in FIG. 7. In the example of FIG. 8, the lid 426 is taller so that there is a gap between the top of the neck 436 of the bottle 406 and the top of the interior of the lid 426. In some examples, as in the example of FIG. 7, the top of the neck 336 of the bottle 306 contacts the top of the interior of the lid 326 when the lid 326 is placed onto the bottle 306, in other examples, as in the example of FIG. 8, there is a gap between the top of the interior of the lid 426 and the top of the interior of the neck 436 of the bottle 406.

As in the example of FIG. 7, in the example of FIG. 8, the shape of the upper surface 428 of the lid 426 is complementary to the shape of the bottom surface 444 of the bottle 406 as to provide greater stability to the breastmilk storage system 420. However, in other configurations, the shapes of the upper surface 428 of the lid 426 and the shape of the bottom surface 444 of the bottle 406 may differ as to provide gaps between the two surfaces when the top container 422a is placed on top of the bottom container 422b. In the example of FIG. 8, the top of the top container 422a is not shown. In some examples, the top container 422a also includes a lid 426. By using the lid 426 with the top container 422a, a user can better protect against spilling of the breastmilk if the top container 422a were to fall or be knocked off of the bottom container 422b of the breastmilk storage system 420. However, in other examples, the top container 422a can be placed on top of the bottom container 422b without the lid 426.

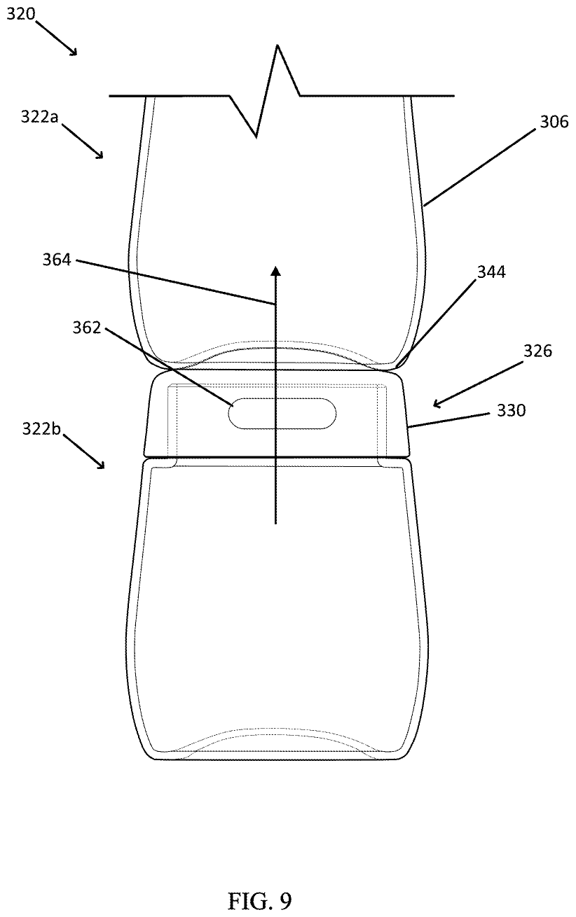

FIG. 9 is a schematic view of the example passive attachment breastmilk storage system 320 of FIG. 7. In this example, the lid 326 is seen with a label 362 that indicates the name of the manufacturer of the breastmilk storage system 320. In some examples, the label 362 may be adhesively affixed to the lid 326. In other examples, the label 362 may be engraved, embossed, laser marked, stamped, etched, or otherwise applied to the lid 326. In some examples, the label 362 includes other information, such as information regarding the contents of the container 322. Although, in the example of FIG. 9, the label 362 appears affixed to the side surface 330 of the lid 326, in other examples, the label 362 may be applied to other surfaces on the lid 326, or on the bottle 306 of the container 322.

FIG. 9 also depicts an example directional pull arrow 364. In some embodiments, the directional pull arrow 364 corresponds to the direction a user pulls the top container 322a to remove it from the bottom container 322b of the breastmilk storage system 320. However, a user need not always pull the top container 322a in the exact direction of the directional pull arrow 364. In some examples, the user can remove the top container 322a by pushing the top container 322a to the side or turning the top container 322a on its side.

In other examples, the directional pull arrow 364 corresponds to the direction the user pulls the lid 326 of the bottom container 322b to remove it from the bottle 306 of the bottom container 322b. In some examples, the user may need to twist the lid 326 of the container 322 before pulling it off of the bottle 306 in the direction of the directional pull arrow 364.

FIG. 10 is another example container 522 that can be used with a passive attachment breastmilk storage system 520. The example of FIG. 10 includes a gripping groove 566 extending around the perimeter of the side of the lid 526. In some examples, the gripping groove 566 assists a user in gripping the lid 526 of the container 522 when moving the container 522 or removing the lid 526 from the bottle 506.

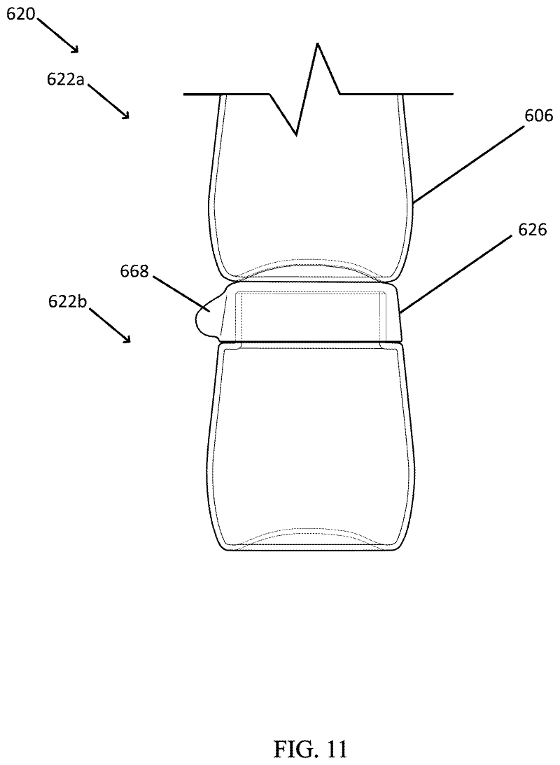

In another embodiment of the example of FIG. 11, the lid 626 of the bottom container 622b includes a hinge 668. The hinge 668 juts out from the side of the lid 626 and facilitates the removal of the lid 626. In the example of FIG. 11, after removing the top container 622a from the lid 626 of the bottom container 622b, a user may push up on the side of the lid 626 opposite the hinge 668. The lid 626 may then be rotated upwards and removed from the neck 636 of the bottle 606 so that the interior 640 of the bottle 606 may be accessed. After removal of the lid 626, the hinge 668 serves to keep the lid 626 attached to the bottle 606 so that the lid 626 may be easily replaced and is not lost by a user.

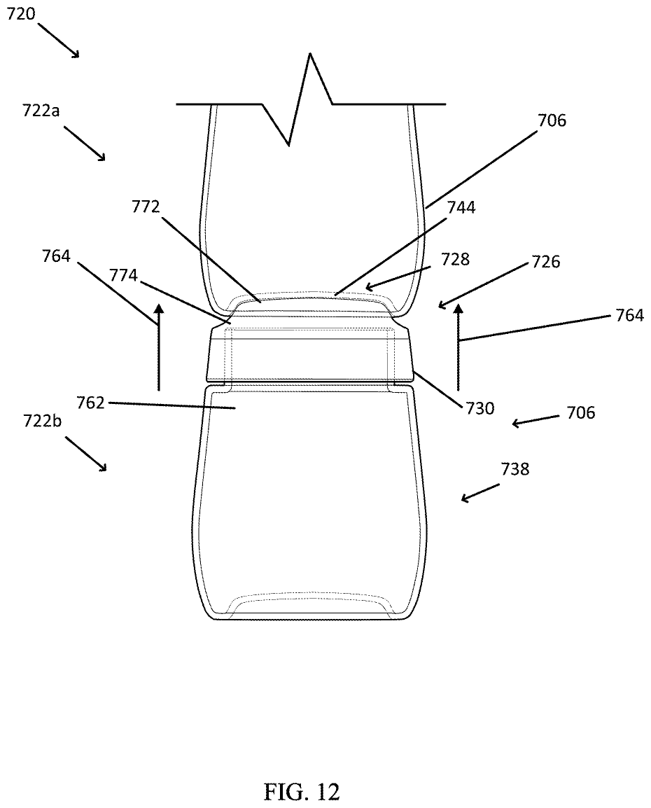

FIG. 12 illustrates another embodiment of an example passive attachment breastmilk storage system 720. The lid 726 of FIG. 12 includes a side surface 730 and a upper surface 728. However, in the example of FIG. 12, the upper surface 728 of the lid 726 is shaped in an alternative format. The upper surface 728 of the lid 726 includes an upper cap 772, and a rounded edge 774. In the example of FIG. 12, the upper cap 772 is rounded in a convex manner, similar to the dome shaped upper surface 328 of FIG. 7. However, unlike FIG. 7, the dome shaped upper surface 728 of the example of FIG. 12, the upper cap 772 does not extend all the way to the edges of the lid 726 and contact the side surface 730 of the lid. Rather, the upper cap 772 contacts the rounded edge 774. The rounded edge 774 connects the upper cap 772 to the side surface 730 of the lid 726. In the example of FIG. 12, the rounded edge 774 is a rounded interior corner that extends around the perimeter of the lid 726 between the upper cap 772 and the side surface 730 of the lid 726. In some examples, the rounded edge 774 may resemble a chamfered edge, similar to the example of FIG. 6.

As noted previously, in the example of FIG. 12, the bottom surface 744 of the bottle 706 of the top container 722a is shaped complementary to the upper surface 728 of the lid 726 of the bottom container 722b. Accordingly, the bottom surface 744 of the bottle of the top container 722a recedes upwards into the body of the bottle 706. The mating surfaces and complementary edges provide enhanced stability, as the greater complexity of the surfaces engaging with each other provides greater surface area for surface adhesion.

As in the example of FIG. 9, the example of FIG. 12 also includes directional pull arrows 764, indicating the direction a user may pull the top container 722a or lid 726 to facilitate easy removal of the top container 722a from the bottom container 722b or the lid 726 from the bottle 706.

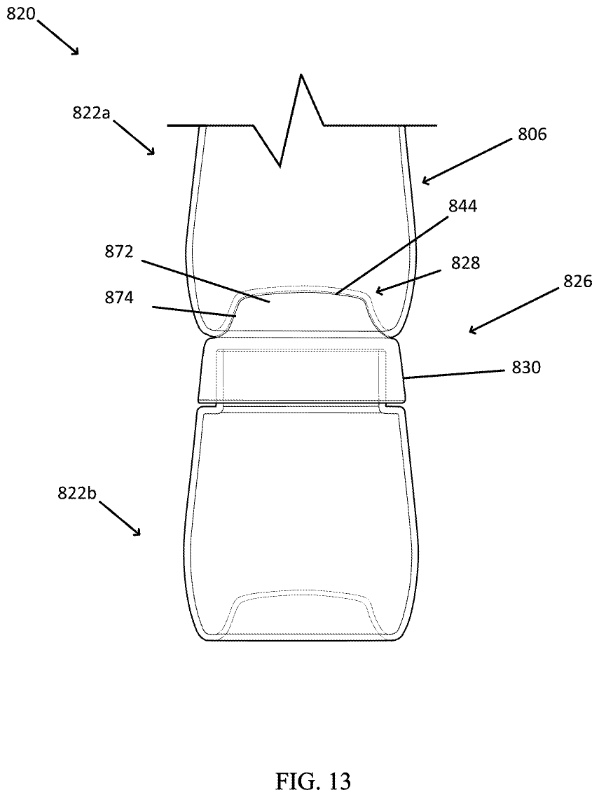

FIG. 13 illustrates another embodiment of an example passive attachment breastmilk storage system 820. The example of FIG. 13 includes a lid 826 with a side surface 830 and an upper surface 828. As in the example of FIG. 12, the upper surface 828 of the lid 826 is shaped in an alternative format and includes the upper cap 872 and the rounded edge 874. However, in the example of FIG. 13, the rounded edge 874 extends further upward than the example depicted in FIG. 12. In some embodiments, the larger rounded edge 874 creates greater stability in the breastmilk storage system 820, as it provides a larger and more complex surface for engaging with the bottom surface 844 of the bottle 806.

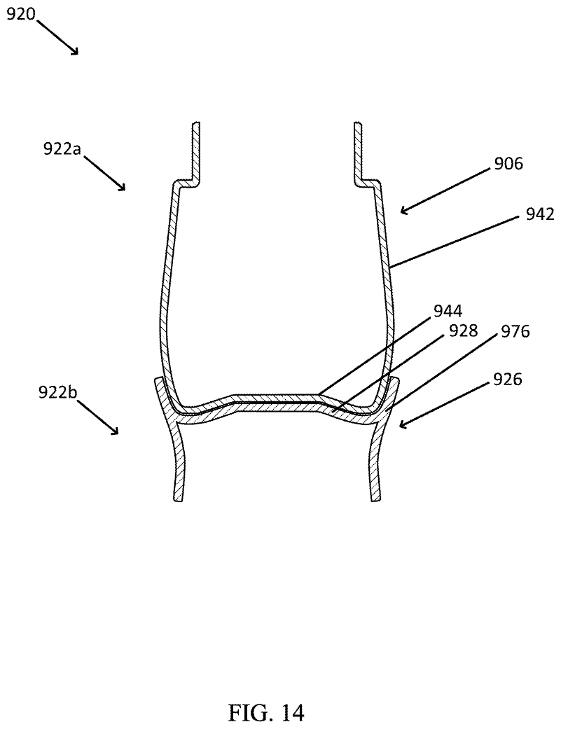

FIG. 14 is a cross-sectional view of a portion of an example breastmilk storage system 920, depicting another example passive attachment between the bottle 906 of the top container 922a and a lid 926 of the bottom container 922b. As depicted in FIG. 14, the bottom surface 944 of the bottle 906 is placed on top of the upper surface 928 of the lid 926. Like in the example system of FIG. 6, the system of FIG. 14 includes a complementary shaped bottom surface 944 of the bottle 906 and upper surface 928 of the lid 926. In contrast to the breastmilk storage system 920 of FIG. 6, however, the lid 926 of the example container 922a of FIG. 14 includes a retaining flange 976. The retaining flange 976 extends upwardly around the perimeter of the upper surface 928 of the lid 926. In the example of FIG. 14, the retaining flange 976 and upper surface 928 are formed to match the shape of the bottom surface 944 of the bottle 906. Thus, when the bottom surface 944 of the bottle 906 is placed on top of the upper surface 928 of the lid 926, the retaining flange 976 surrounds and contacts a portion of the side surface 942 of the bottle 906 near the bottom of the bottle 906.

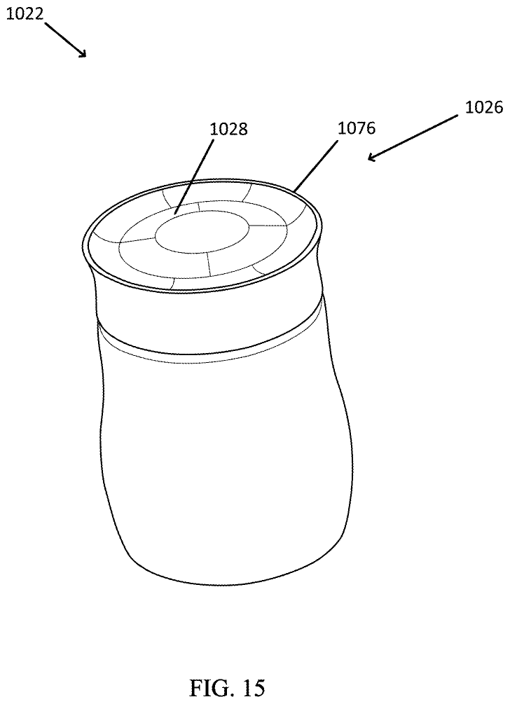

FIG. 15 is a perspective view of a container 1022 of another example passive attachment breastmilk storage system 1020. The container of FIG. 15 includes a lid 1026 with a retaining flange 1076. In the example of FIG. 15, the retaining flange 1076 extends above the uppermost point of the upper surface 1028 of the lid 1026, however, in other examples, the retaining flange 1076 extends only partially upwards and does not extend above the uppermost point of the upper surface 1028 of the lid 1026. In the example of FIG. 15, the center of the upper surface 1028 of the lid 1026 is flattened out, however, in other examples, the upper surface 1028 of the lid 1026 is completely dome shaped.

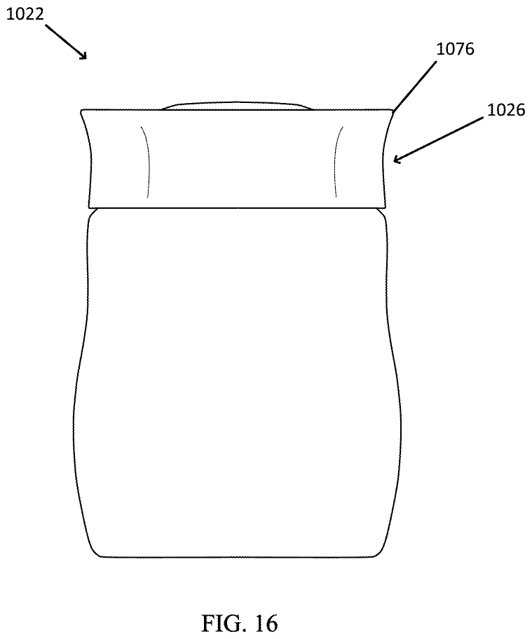

FIG. 16 is a front view of the example container 1022 of FIG. 15. In the example of FIG. 16, the retaining flange 1076 is shown to flare outward from the center of the lid 1026.

FIGS. 17 and 18 depict alternative cross-sectional views of the example container 1022 of FIG. 15. In the example of FIGS. 17 and 18, the lid 1026 and bottle 1006 include the engagement features 1046 for securing the lid 1026 to the bottle 1006. The neck 1036 of the bottle 1006 includes lid engagement features 1048 and the interior of the lid 1026 contains bottle engagement features 1052. The lid engagement features 1048 and bottle engagement features 1052 and formed to engage with each other as the lid 1026 is placed onto the bottle 1006. The engagement features 1046 secure the lid 1026 to the bottle 1006 and prevent the lid 1026 from being dislodged as the container 1022 is moved around. Thus, the breastmilk contained within the container 1022 can be securely held within the interior space of the bottle 1006 without spilling therefrom. In some examples, the engagement features 1046 consist of one or more ribs, in which case the lid 1026 snaps onto the bottle 1006 using an interference fit. In other examples, the engagement features 1046 are threads. When threads are used, the bottle engagement features 1052 of the lid 1026 can engage with and thread onto the lid engagement features 1048 of the bottle 1006 as the lid 1026 is twisted onto the neck 1036 of the bottle 1006.

In the examples of FIGS. 17 and 18, the curved side surface 1030 of the lid 1026 is depicted, and the retaining flange 1076 is seen to gradually curve and flare upwards and out from the center of the lid 1026.

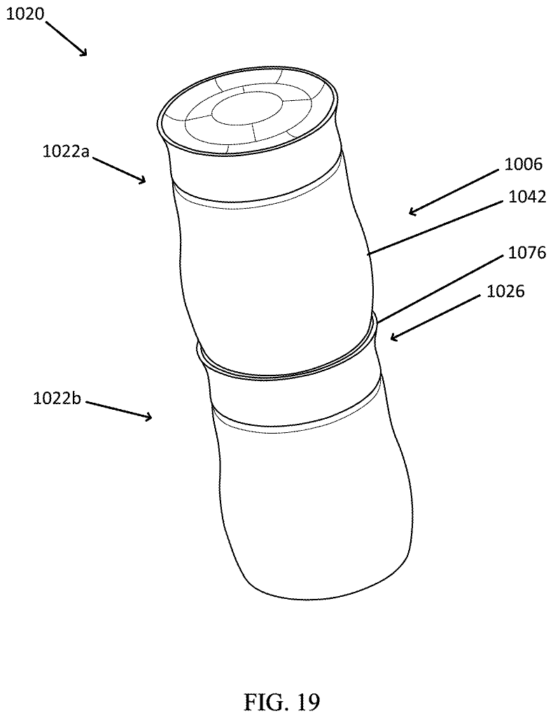

FIG. 19 depicts a perspective view of an example passive attachment breastmilk storage system 1020 utilizing the example containers 1022a,b of FIGS. 15-18. As depicted in FIG. 19, a top container 1022a is placed on top of the lid 1026 of a bottom container 1022b. The bottom container 1022b includes a retaining flange 1076, which extends around and contacts the bottom of the side surface 1042 of the bottle 1006 of the top container 1022a. The retaining flange 1076 functions to secure the bottle 1006 of the top container 1022a in place on top of the lid 1026 of the bottom container 1022b.

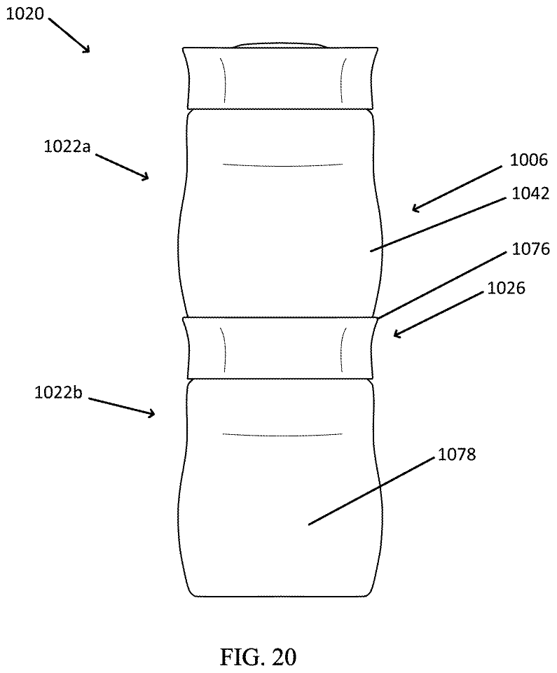

FIG. 20 is a front view of the example breastmilk storage system 1020 of FIG. 19. As shown in the example of FIG. 20, the retaining flange 1076 extends partially up the side surface 1042 of the bottle 1006 of the top container 1022a, however, in other examples, the retaining flange 1076 extends further up the side surface 1042 of the bottle 1006 of the top container 1022a. In the example of FIG. 20, the example breastmilk storage system 1020 utilizes containers 1022 with opaque bottles with graduations 1078 marked on the side surface 1042 of the bottles.

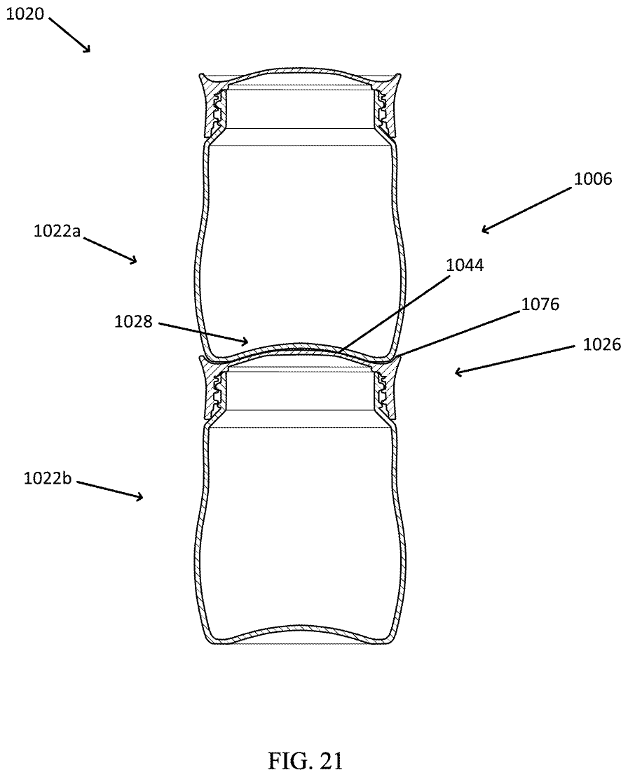

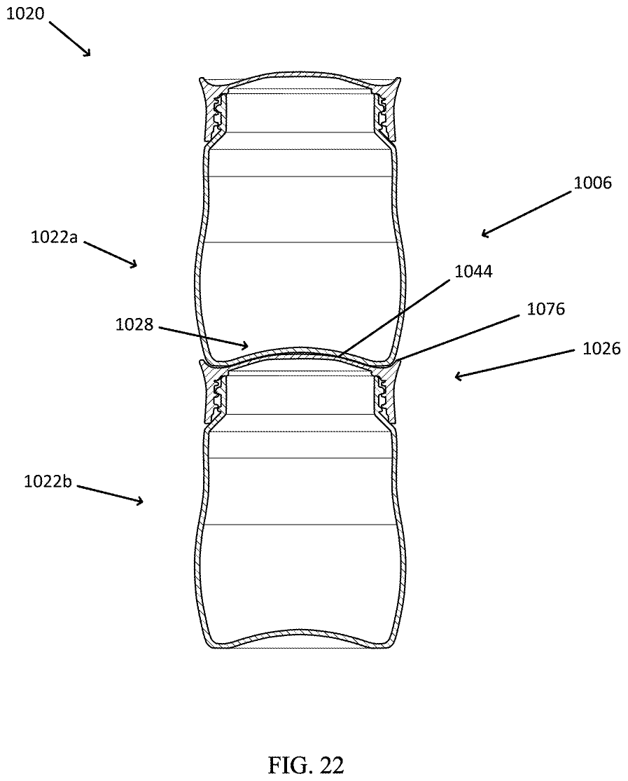

FIGS. 21 and 22 each depicts cross-sectional views of the example passive attachment breastmilk storage system 1020 of FIGS. 19 and 20. The breastmilk storage system 1020 of FIGS. 21 and 22 includes a top container 1022a stacked on top of a bottom container 1022b. The bottom surface 1044 of the bottle 1006 of the top container 1022a is shaped complementary to the upper surface 1028 of the lid 1026 of the bottom container 1022b. The upper surface 1028 of the lid 1026 of the bottom container 1022b includes a dome shape with a rounded peak in its center. The rounded peak extends upwardly above the top of the retaining flange 1076. In the example of FIGS. 21 and 22, the top and bottom containers 1022a,b include identical bottles 1006 and identical lids 1026, however, in other embodiments, the shape of the lids 1026 and bottles 1006 may differ between the top container 1022a and bottom container 1022b.

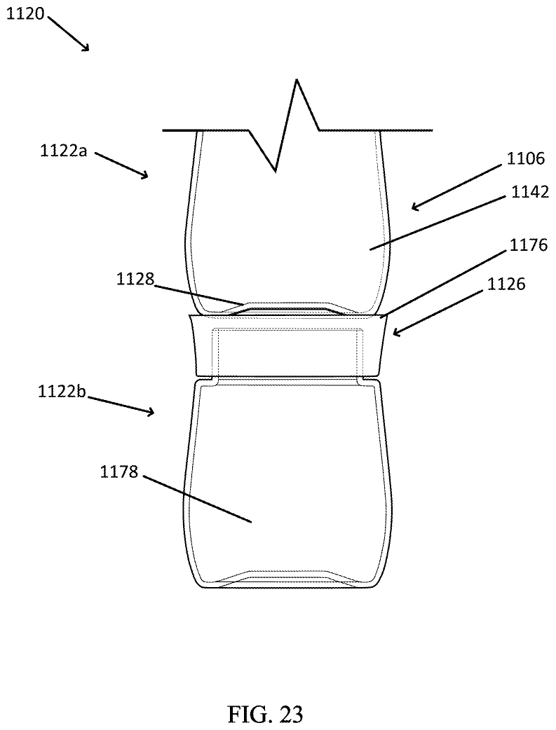

FIG. 23 is a semi-transparent front view of a portion of another example passive attachment breastmilk storage system 1120. In the example of FIG. 23, the breastmilk storage system 1120 includes a bottom container 1122b with a lid 1126. The lid 1126 of the bottom container 1122b includes a retaining flange 1176, and an upper surface 1128. The upper surface 1128 of the lid 1126 includes a flat center that extends above the uppermost portion of the retaining flange 1176. In the example of FIG. 23, the retaining flange 1176 only extends upwardly to cover the bottom edge of the side surface 1142 of the bottle 1106 of the top container 1122a. The example breastmilk storage system 1120 utilizes containers 1122 with transparent bottles 1106 so that the contents of the container 1122 can easy be seen. The bottles 1106 also each include a pair of graduations 1178 on the side surfaces 1142, which allows for easy measurement of the volume of the liquid contends of the containers 1122.

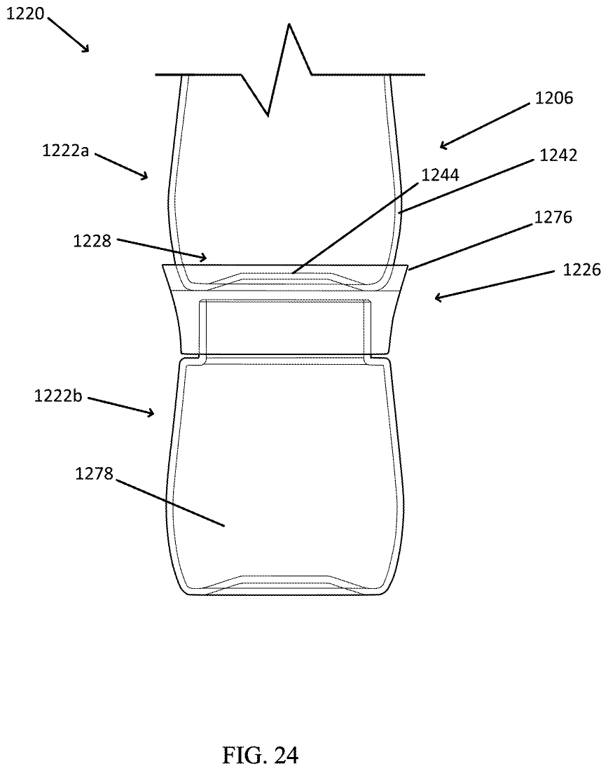

FIG. 24 is semi-transparent front view of a portion of another example passive attachment breastmilk storage system 1220. The breastmilk storage system 1220 of FIG. 24 is similar to the breastmilk storage system 1120 of FIG. 23, insofar as both examples include a lid with a flat center upper surface. In FIG. 24, however, the retaining flange 1276 is configured to extend upwardly above the upper surface 1228 of the lid 1226. Thus, in this example, the retaining flange 1276 extends further up the side surface 1242 of the of the bottle 1206 of the top container 1222a than in the example of FIG. 23. In some situations, the enhanced retaining flange 1276 of FIG. 24 provides greater stability to the bottle 1206 of the top container 1222a, as it provides a greater engagement surface area between the upper surface 1228 of the lid 1226 and the bottom surface 1244 of the bottle 1206. For example, using the enhanced retaining flange 1276 of FIG. 24 may make it more difficult for the top container 1222a to be tipped off of its position on top of the bottom container 1222b.

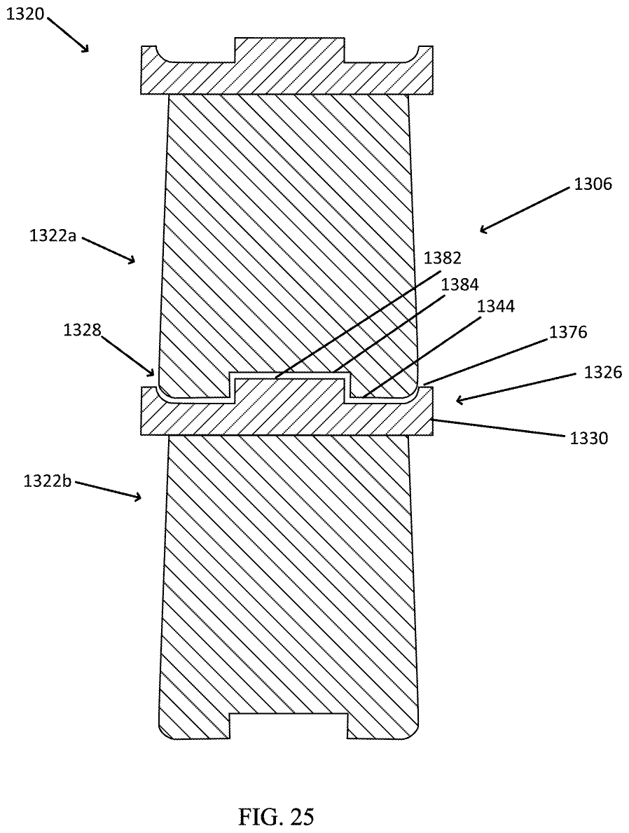

FIG. 25 is cross-sectional view of another example passive attachment breastmilk storage system 1320. The breastmilk storage system 1320 includes a top container 1322a stacked on top of a bottom container 1322b. The top container 1322a and the bottom container 1322b each include an identically shaped lid 1326 and bottle 1306. The lids 1326 of the top container 1322a and bottom container 1322b include a side surface 1330, a retaining flange 1376, and an upper surface 1328. The upper surface 1328 of the lid 1326 further includes a protrusion 1382. The side surface 1330 of the lid 1326 extends vertically straight downward, however, in other examples, the side surface 1330 may be positioned at an angle or may have a curved profile. The side surface 1330 is configured to extend vertically upward until it reaches the retaining flange 1376. The retaining flange 1376 includes a horizontal top that is squared off at a ninety-degree angle from the side surface 1330. The retaining flange 1376 also includes a curved portion that curves downwards and connects with the upper surface 1328 of the lid 1326. The upper surface 1328 of the lid 1326 is generally flat aside from the protrusion 1382 positioned at its center. The protrusion 1382 is a cylindrical feature that extends vertically upward from the upper surface 1328. Although the protrusion 1382 is squared off from the upper surface 1328 of the lid 1326 in FIG. 25, in other examples, the protrusion 1382 may gradually slope or angle upward from the upper surface 1328. Likewise, in other examples, the protrusion 1382 may define other shapes, such as, for example, a triangle, a square, an oval, or any polygon. In the example of FIG. 25, the protrusion 1382 is squared off at its top to create a flat surface, however, in some examples, the protrusion 1382 has a curved or pointed upper surface. Likewise, although in the example of FIG. 25 the protrusion 1382 extends above the top of the retaining flange 1376, in other embodiments, the protrusion 1382 may terminate below the top of the retaining flange 1376.

Again, the bottom surface 1344 of the bottle 1306 of the top container 1322a is configured to match the shape of the upper surface 1328 of the lid 1326 of the bottom container 1322b. In this embodiment, the bottom surface 1344 includes a rounded edge, around which the curved portion of the retaining flange extends. The bottom surface 1344 also includes a recess 1384, configured to match the shape of the protrusion 1382 of the upper surface 1328 of the lid 1326. In some embodiments, different bottles 1306 contain different shaped recesses 1384 so that only certain bottles 1306 may be stacked on top of a given lid 1326. Likewise, in some embodiments, different lids 1326 contain different shaped protrusions 1382, so that only some lids 1326 may be stacked under a given bottle 1306.

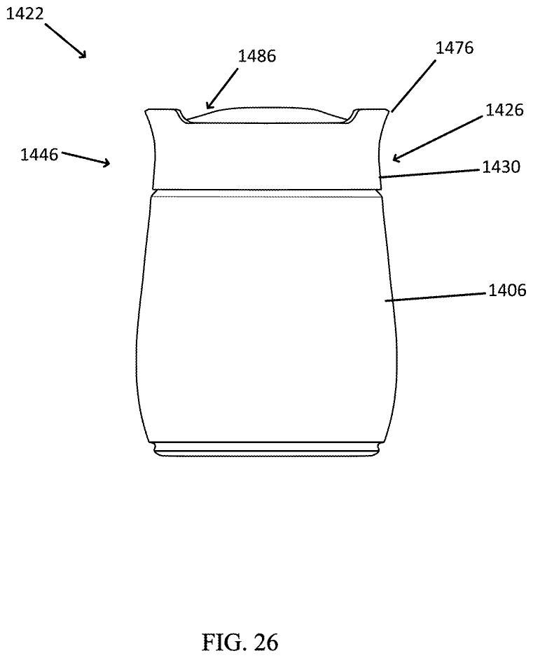

FIG. 26 is a front view of another example container 1422 used in conjunction with another example passive attachment breastmilk storage system 1420. In the example of FIG. 26, the container 1422 includes lid 1426 with a retaining flange 1476. However, in the example of FIG. 26, the retaining flange 1476 is discontinuous around the perimeter of the lid 1426. In the example of FIG. 26, the retaining flange 1476 includes at least two gaps 1486, however, in some examples, the retaining flange 1476 may include a different number of gaps 1486. Although the gaps 1486 in the retaining flange 1476 of FIG. 26 appear to extend the length of most of the diameter of the lid 1426, in some examples, the gaps 1486 are larger or smaller. In some examples, the gaps 1486 are merely slits in the retaining flange 1476. In the example of FIG. 26, the retaining flange 1476 is biased to curve or flare outward from the middle of the lid 1426.

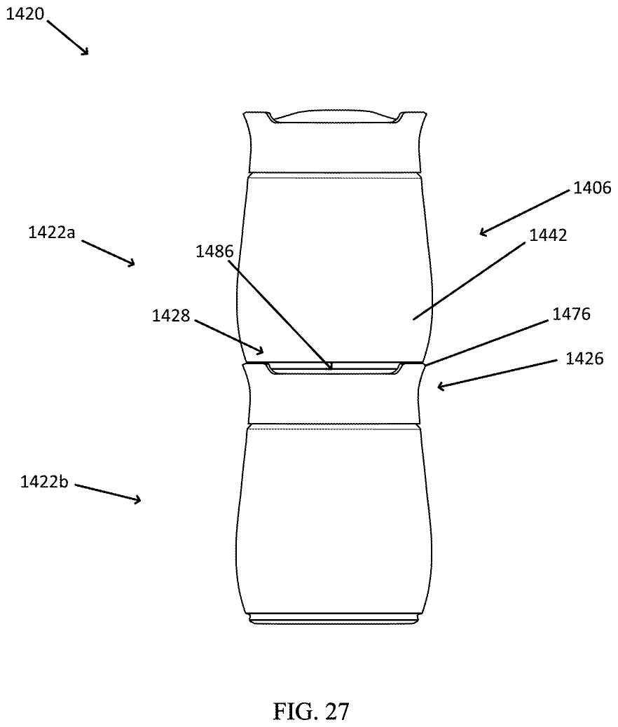

FIG. 27 depicts a front view of an example passive attachment breastmilk storage system utilizing the example containers 1422 of FIG. 26. As seen in the example of FIG. 27, the containers 1422 include a lid 1426 with gaps 1486 in the retaining flange 1476. In some examples, the gaps 1486 allow the top and bottom containers 1422a,b stacked in a passive attachment breastmilk storage system 1420 to be more easily separated than in a passive attachment breastmilk storage system 1420 where there are no gaps 1486 included in the retaining flange 1476. However, in some examples, the inclusion of the gaps 1486 comes at the expense of stability in the passive attachment breastmilk storage system 1420 when the containers 1422a,b are stacked.

FIG. 28 depicts a front view of another example passive attachment breastmilk storage system 1520, similar to the embodiment of FIG. 27. As in the embodiment of FIG. 27, the example of FIG. 28 depicts containers 1522 that include a lid 1526 with gaps 1586 in the retaining flange 1576. In contrast to the example of FIG. 27, in the example of FIG. 28, the breastmilk storage system includes containers 1522 with clear bottles 1506.

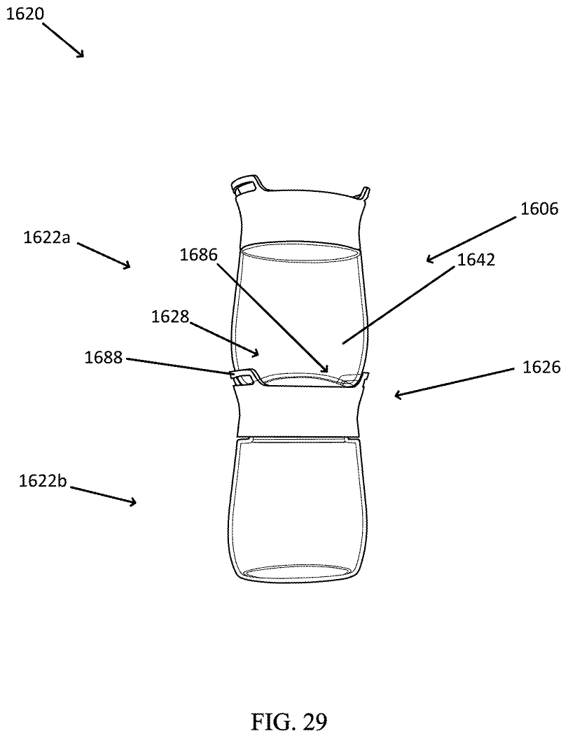

FIG. 29 is a front view of another example passive attachment breastmilk storage system 1620. In the example of FIG. 29, the containers 1622 include lids 1626 with retaining handles 1688. Like the retaining flange 1676 discussed above, the retaining handles 1688 extend upwardly from the upper surface 1628 of the lid 1626 above the uppermost point of the upper surface 1628. The retaining handles 1688 flare out from the middle of the lid 1626, however, in other examples, the retaining handles 1688 may point in other directions. The example containers 1622 of FIG. 29 include lids 1626 with two retaining handles 1688 each, however, in other examples, different numbers of retaining handles 1688 may be included. In the example of FIG. 29, each retaining handle 1688 extends partway around the perimeter of the lid 1626 and include gaps 1686 along the edge of the lid 1626 between them. Each of the retaining handles 1688 also contains a space in between a handle of the retaining handle 1688 and the edge of the lid 1626. In different examples, different sized retaining handles 1688 or gaps 1686 may be used. In some examples, the retaining handles 1688 provide stability to the containers 1622 placed on top of the lid 1626. In other examples, the retaining handles 1688 provide benefits to a user, such as providing a user a space with which to grasp the lid 1626, allowing the user to attach a lanyard to the lid 1626, or providing a leverage point for a user to detach the lid 1626 from the bottle 1606.

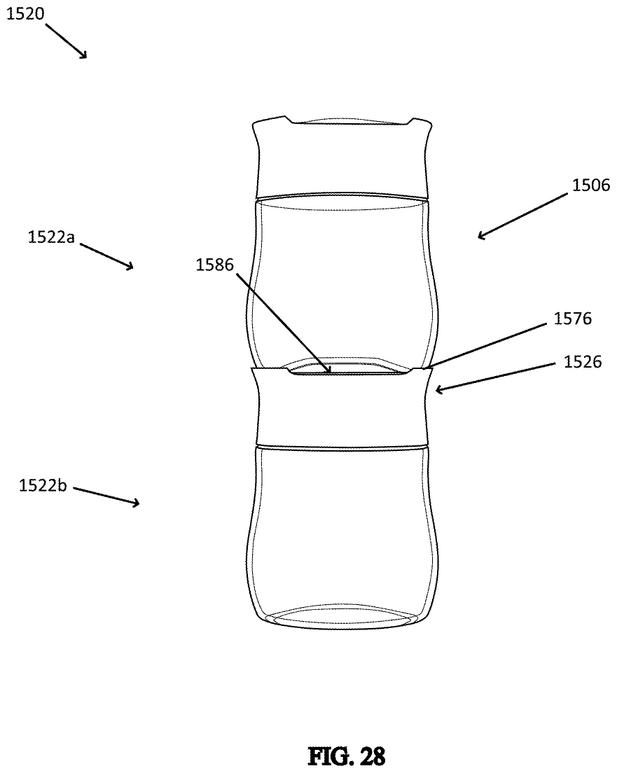

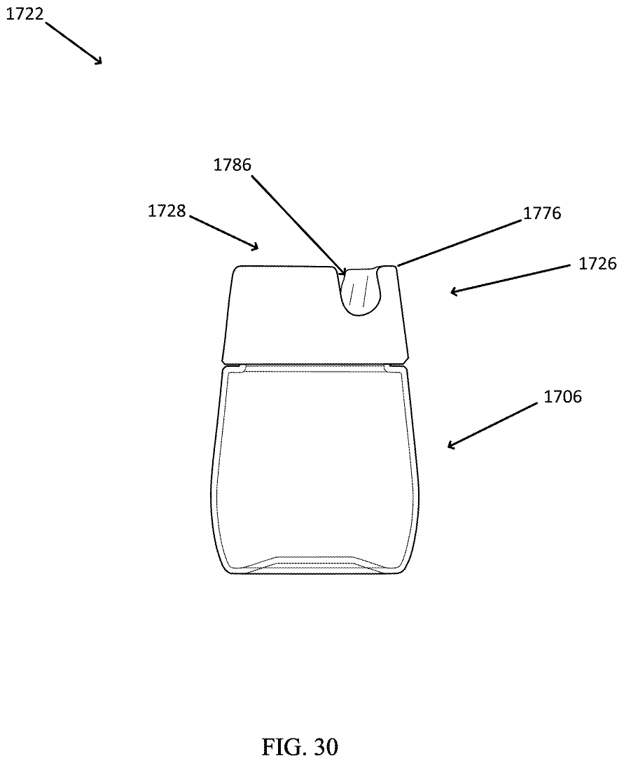

FIG. 30 is another example container 1722 that can be used with the passive attachment breastmilk storage system 1720. In the example of FIG. 30, the lid 1726 of the container 1722 includes a retaining flange 1776 with two gaps 1786, however, in other examples, the retaining flange 1776 may include different numbers of gaps 1786. In the example of FIG. 30, the retaining flange 1776 extends further upwardly than the retaining flange 1776 of the other lids 1726 in FIGS. 26-28. Also, in this example, the retaining flange 1776 is not biased to flare outwards, but rather, extends upwardly in a direction slightly towards the middle of the lid 1726.

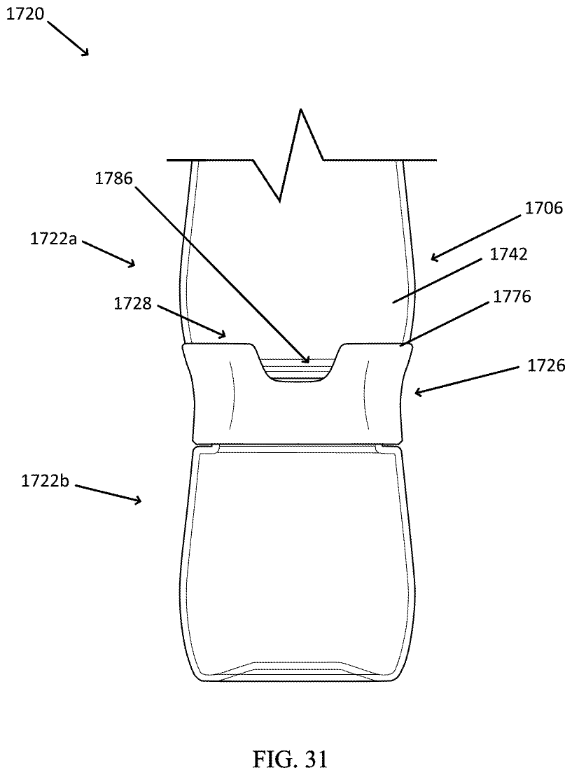

FIG. 31 is an example passive attachment breastmilk storage system 1720 using the containers 1722 of FIG. 30. In this example, the retaining flange 1776 is made from a flexible material and is manipulated by the side surfaces 1742 of the bottle 1706 of the top container 1722a to flare outwardly with respect to the middle of the lid 1726. Because the retaining flange 1776 is not biased to flare outwardly, the retaining flange 1776 tends to recede back towards its middle facing position shown in FIG. 30. However, since the top container 1722a is positioned on the upper surface 1728 of the lid 1726, the side surface 1742 of the bottle 1706 prevents the retaining flange 1776 from receding all the way back to its neutral position. Thus, the retaining flange 1776 rests against the side surface 1742 of the bottle 1706 and provides stability to the container 1722a stacked on top of the lid 1726. In some examples, the pressure applied by the retaining flange 1776 against the side surface 1742 of the bottle 1706 provides a suction effect that holds the container 1722a in place on top of the lid 1726. In these examples, the user may release the top container 1722a from the bottom container 1722b by peeling the edges of the retaining flange 1776 away from the side surfaces 1742 of the bottle 1706.

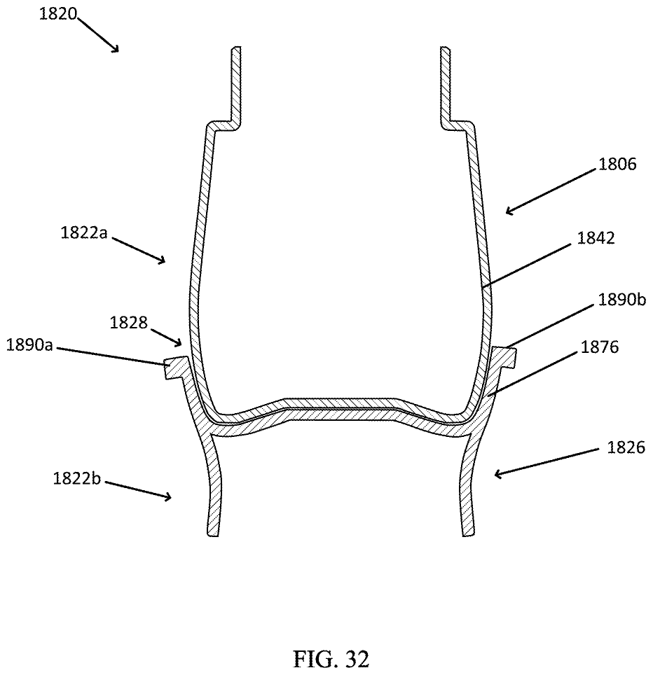

FIG. 32 is another cross-sectional view of a portion of an example passive attachment breastmilk storage system 1820. In the example of FIG. 32, the lid 1826 of the bottom container 1822b includes a retaining flange 1876. The retaining flange 1876 extends around the circumference of the lid 1826 and is made from a flexible material. When the bottle 1806 of the top container 1822a is placed onto the lid 1826 of the bottom container 1822b, the retaining flange 1876 is pushed outwardly by the side surface 1842 of the bottle 1806 and rests against the side surface 1842 of the bottle 1806. In some examples, when resting against the side surface 1842 of the bottle 1806, the retaining flange 1876 provides a suction effect that holds the bottle 1806 in place. In some examples, the material of the lid enhances this suction effect. This effect may be observed, for example, when the lid 1826 and retaining flange 1876 are manufactured using an elastomeric material. When providing the suction effect, the lid 1826 of the bottom container 1822b secures the top container 1822a so that it cannot be easily removed by lifting the top container 1822a off of the bottom container 1822b. Thus, the retaining flange 1876 also includes release tabs 1890a,b. The release tabs 1890a,b are positioned on the upper edge of the retaining flange 1876 and provide a surface that the user can grasp to release the suction of the retaining flange 1876 against the side surface 1842 of the bottle 1806. In some examples, the release tabs 1890a,b extend around the entire perimeter of the lid 1826, however, in other examples, the release tabs 1890a,b are only disposed at separate locations around the edge of the lid 1826. The release tabs 1890a,b can be made of a variety of materials such as, for example, rubber or plastic.

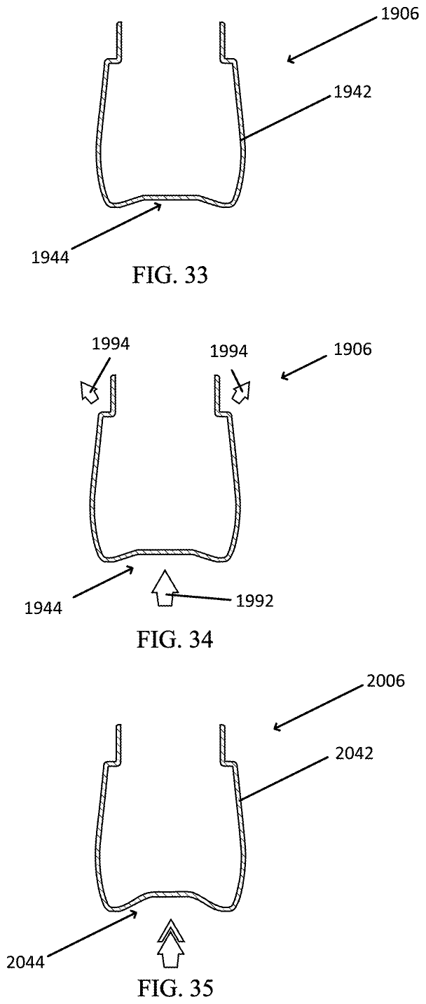

FIGS. 33 and 34 depict a cross-sectional view of an example bottle 1906 shapes that may be used with various embodiments of the passive attachment breastmilk storage system described herein. In the example of FIG. 33, a bottle 1906 is shown with a moderately concaved bottom surface 1944 to accompany a moderately domed upper surface of a lid. The bottom surface 1944 of this example includes a flat surface recessed within the bottom surface 1944. In some examples, this example bottle 1906 is used with a lid that has a flat top on its upper surface such as, for example, the examples of FIG. 23. The bottle 1906 also includes a side surface 1942 that gradually expands outward from the bottom of the side surface 1942. In some examples, the side surface 1942 mates with the curved retaining flange on the lid as depicted in, for example, FIG. 28.

FIG. 34 depicts the example bottle 1906 of FIG. 33 with directional arrows. The lower directional arrow 1992 emphasizes the recess of the bottom surface 1944 of the bottle 1906 that is contacted by the upper surface of a lid. The upper directional arrows 1994 indicate directions that the bottle 1906 may be pulled to remove the bottle 1906 from a lid when the bottle 1906 is used with the passive attachment breastmilk storage system.

FIG. 35 depicts another example bottle that may be used with various embodiments of the passive attachment breastmilk storage system described herein. In the example of FIG. 35, the bottle 2006 is shown with a bottom surface 2044 that has a deeper recess than the example of FIGS. 34 and 35. The recessed bottom surface 2044 of the example of FIG. 35 also includes a flat surface for contacting a flattened top of the domed upper surface of a lid. In the example of FIG. 35, the bottle 2006 is compatible with lids that include a taller domed upper surface, such as, for example, the example of FIG. 13. However, the deeper recess of bottom surface 2044 of the example bottle 2006 of FIG. 35 may also be used with lids that have shorter domed upper surfaces. In such cases, a portion of the upper surface of the lid may not contact the flat surface within the recess of the bottom surface 2044.

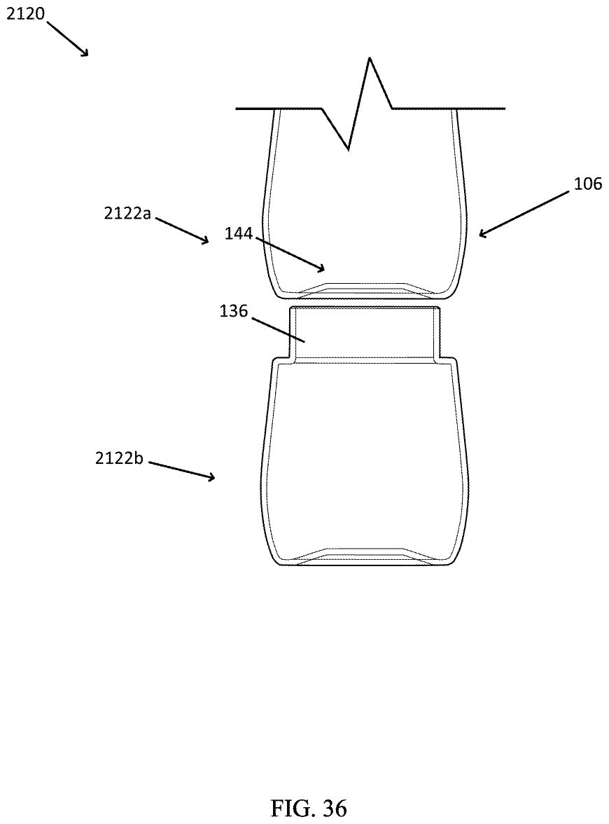

FIG. 36 is an example of another passive attachment breastmilk storage system 2120 using the bottle 106 of FIG. 5, without any lids. Generally, the lid assists in providing stability to the breastmilk storage system. In the example of FIG. 36, a bottle 106 of a top container 2122a and a bottle 106 from a bottom container 2122b are stacked on top of each other. However, in this example, the bottom container 2122b is missing a lid. Because the bottom container 2122b does not have a lid, the bottle 106 of the top container 2122a is placed on the neck 136 of the bottle 106 of the bottom container 2122b. The neck 136 is cylindrical in shape and therefore, is not shaped complementary to the bottom surface 144 of the bottle 106 of the top container 2122a. Thus, when the top container 2122a is placed on the bottle 106 of the bottom container 2122b, the top container 2122a may easily fall or be knocked off of its position on top of the bottom container 2122b. Using the lids previously described, the unstable effect produced by stacking the bottles 106 directly upon each other can be mitigated.

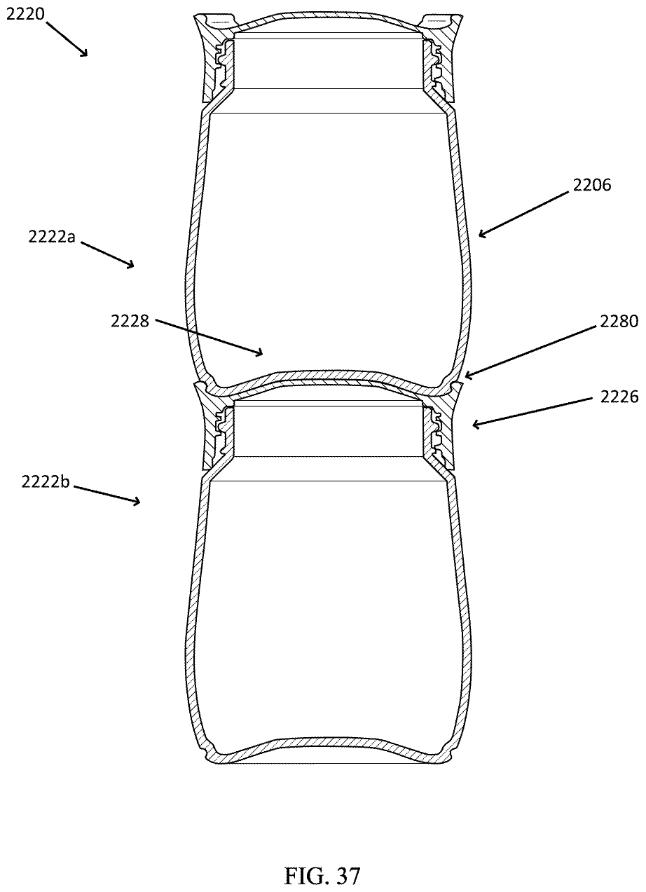

FIGS. 37-44 depict another aspect of the present invention. FIG. 37 is a cross-sectional view of an example active attachment breastmilk storage system 2220, depicting the active attachment between a bottle 2206 of a top container 2222a and a lid 2226 of a bottom container 2222b. In the example of FIG. 37, the top container 2222a stacks on top of the bottom container 2222b in a similar manner to the passive attachment breastmilk storage systems previously described. However, in the active attachment breastmilk storage system 2220, the lid 2226 of the bottom container 2222b further includes active attachment features 2280 that secure the top container 2222a in its place on top of the bottom container 2222b. In the example of FIG. 37, only two containers 2222 are shown stacked on top of each other. However, in other examples, multiple containers can be stacked on top of each other using different the examples of the active attachment breastmilk storage system.

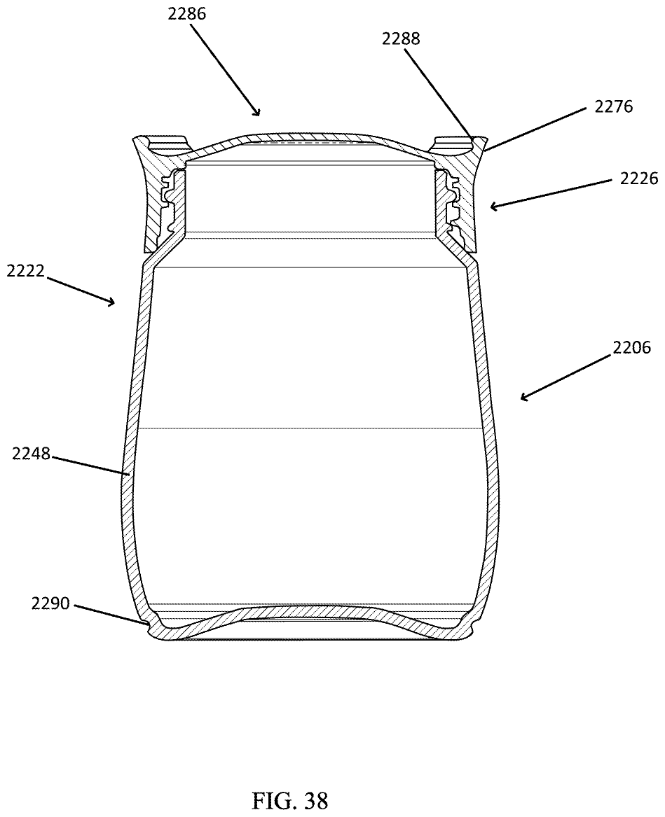

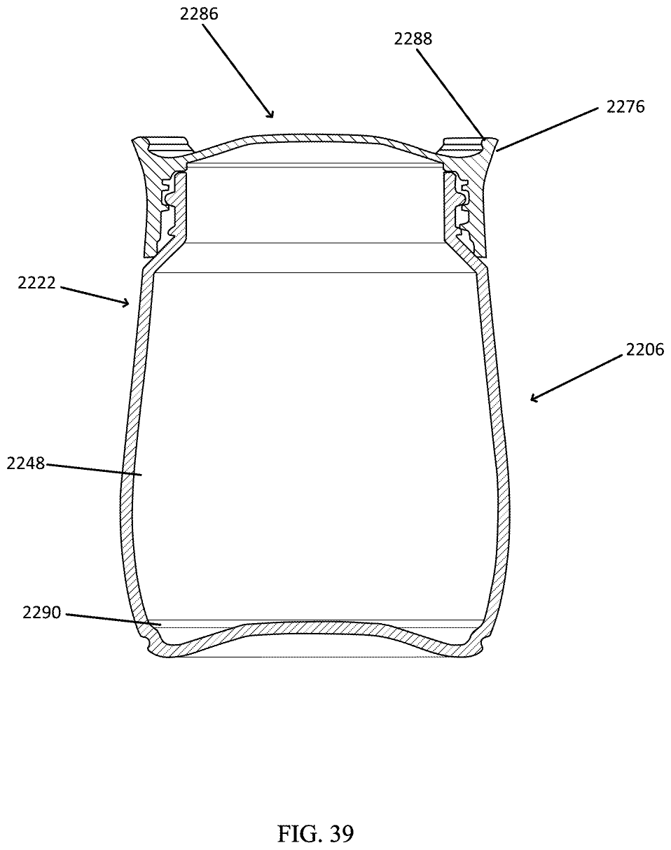

FIGS. 38 and 39 are cross-sectional views of a container 2222 of the active attachment breastmilk storage system of FIG. 37. In the example of FIGS. 38 and 39, the container 2222 includes a lid 2226 and a bottle 2206. The lid 2226 of the container 2222 includes the retaining flange 2276 and also includes active bottle attachment features 2288 along the upper edge of the retaining flange 2276. The active bottle attachment features 2288 of the example of FIG. 38 are ribbed edges, wherein the thickness of the ribbed edge is greater than the thickness of an adjacent lower portion of the retaining flange 2276. In the example of FIGS. 38 and 39, the retaining flange 2276 does not extend all the way around the edge of the lid 2226. Rather, the retaining flange 2276 includes a gap 2286, so that the retaining flange 2276 is broken along the circumference of the lid 2226. In other examples, the retaining flange 2276 is continuous around the perimeter of the lid 2226.

The container 2222 also includes a bottle 2206 with an active lid attachment feature 2290 positioned near the bottom of the side surface 2248 of the container 2222. In the example of FIGS. 38 and 39 the active lid attachment feature 2290 is an undercut groove that extends around the circumference of the side surface 2248 of bottle 2206. In some examples, the groove is continuous, while in other examples, the groove is broken around the bottle 2206 circumference.

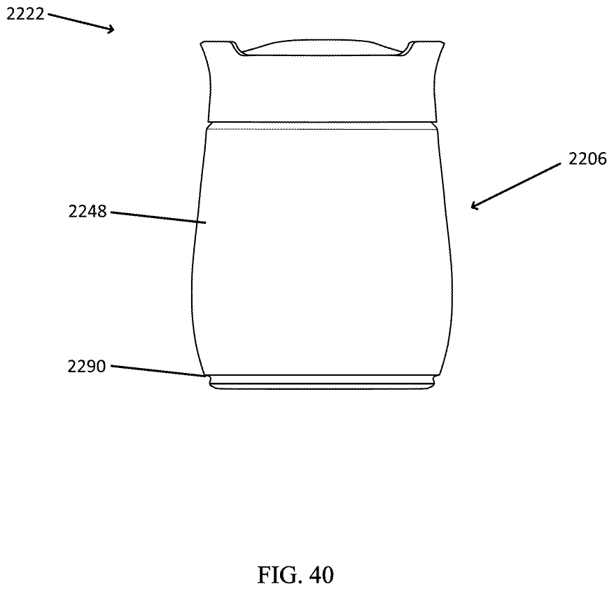

FIG. 40 is a front view of the container 2222 of the active attachment breastmilk storage system 2220 of FIG. 37. As noted above, the bottle 2206 of the container 2222 includes active lid attachment feature 2290 that resembles a continuous groove that extends around the bottom of the side surface 2248 of the container 2222.

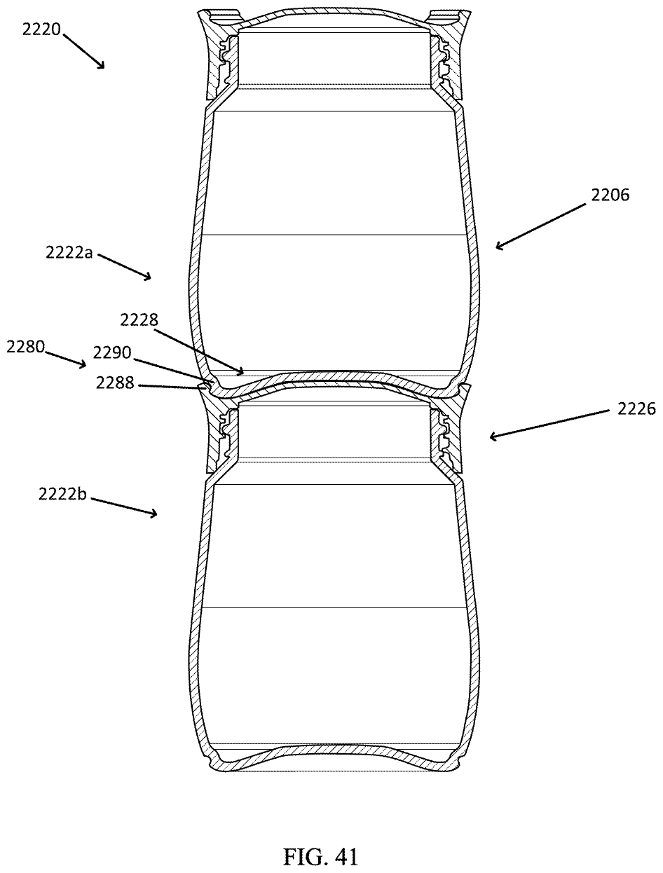

FIGS. 41 and 42 are additional cross-sectional views of the active attachment breastmilk storage system 2220 of FIG. 37. In the examples of FIGS. 41 and 42, the active lid attachment feature 2290 of the bottle 2206 of the top container 2222a is configured to receive the active bottle attachment feature 2288 of the lid 2226. Thus, the ribbed edge of the active bottle attachment feature 2228 fits within the groove of the active lid attachment feature 2290. In some examples, the active lid attachment feature 2290 of the bottle 2206 and the active bottle attachment feature 2288 of the lid 2226 mate together with an interference fit. A user is able to engage the interference fit by pushing the top container 2222a down onto the bottom container 2222b so that the active attachment features 2280 snap into place. In other examples, the active attachment features 2280 are threads. In such examples, the active attachment features 2280 engage with each other when a user places the top container 2222a on top of the bottom container 2222b and twists the top container 2222a in the direction of the threads. The user may then disengage the attachment features 2280 by twisting the top container 2222a the opposite direction. In this example, the retaining flange 2276 of the lid 2226 may be provided with internal threads while the side surface 2242 of the bottle 2206 is provided with matching external threads. In some examples, the threads of the attachment features 2280 are configured to be opposite of the threads of the engagement features 2246 that secure the lid 2226 to its respective bottle 2206. In such cases, a user will not inadvertently detach the lid 2226 from the bottle 2206 when removing the top container 2222a from the bottom container 2222b.

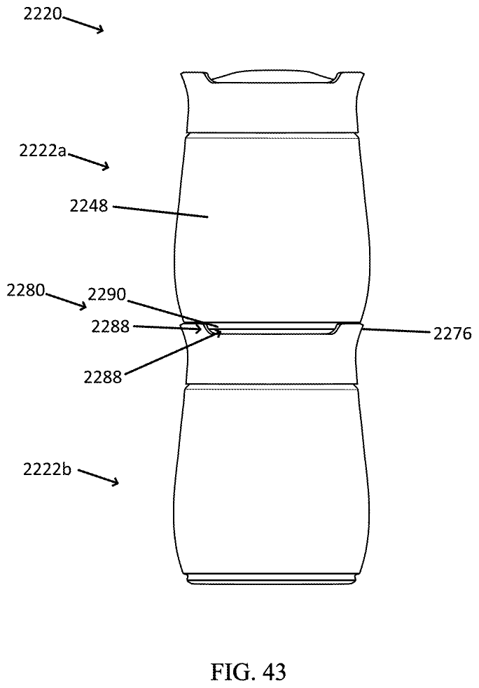

FIG. 43 is a front view of the active attachment breastmilk storage system 2220 of FIG. 37. Although the active lid attachment feature 2290 of the bottle 2206 is continuous around the circumference of the side surface 2248 bottle 2206, the active bottle attachment feature 2288 of the lid 2226 is discontinuous, due to the gaps 2286 in retaining flange 2276 of the lid 2226 that exist around the perimeter of the lid 2226. In some examples, the gaps 2286 make it easier for a user to break the active attachment connection between the top and the bottom containers 2222a,b, as they decrease the amount of engagement area between the attachment features 2280.

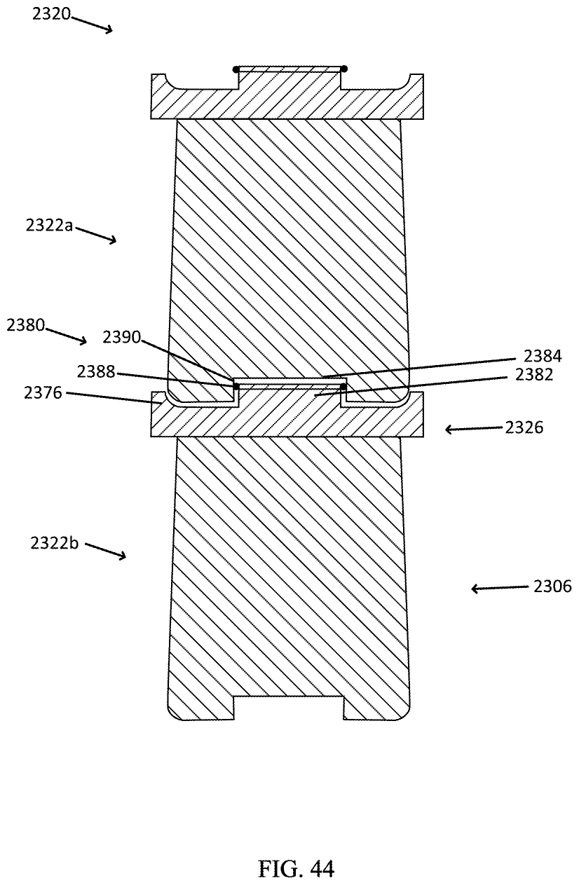

FIG. 44 is a cross-sectional view of another active attachment breastmilk storage system 2320. The example of FIG. 44 is configured similarly to the example passive attachment breastmilk storage system 1320 of FIG. 25. In the example of FIG. 44, the bottle 2306 and lid 2326 include attachment features 2380. In the example of FIG. 44, the protrusions 2382 on the lids 2326 include active bottle attachment features 2388. In some examples, as depicted in the example of FIG. 44, the active bottle attachment features 2388 consist of a rib that 2388 extends as a ring around the perimeter of the entire upper edge of the protrusion 2382. However, in other examples, the active bottle attachment features 2388 extend only partially around the perimeter of the protrusion 2382. The active bottle attachment features 2388 consist of a rib similar to the rib on the retaining flanges 2376 of the lids 2326 of FIGS. 37-44. However, in other examples, the attachment features 2380 may include threads.

Likewise, the recess of the bottles 2306 includes active lid attachment features 2390. In this example, the active lid attachment features 2390 include a groove around the edge of the recess 2384 near the top of the recess 2384. In other examples, the active lid attachment features 2390 include threads. In such cases, the protrusion 2382 of the lid 2326 may be provided with external threads while the recess 2384 of the bottle 2306 is provided with matching internal threads. The active lid attachment features 2390 of the bottle 2306 are configured to engage with the active bottle attachment features 2388 of the lid 2326 to secure the lid 2326 to the bottle 2306 when the top container 2322a is placed on top of the bottom container 2322b.

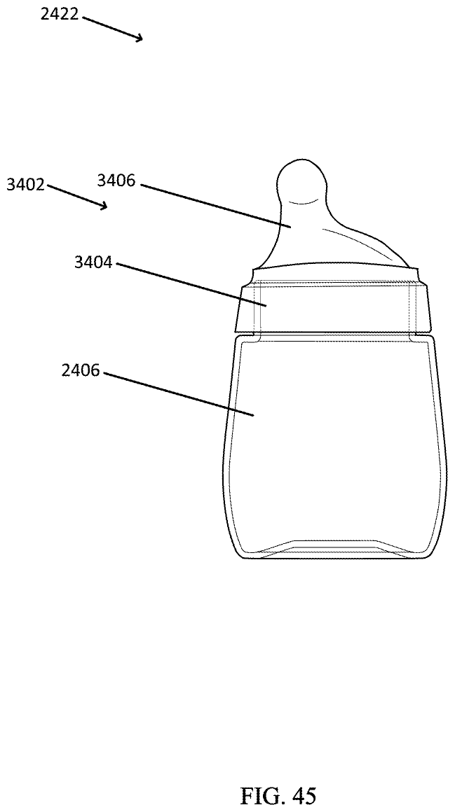

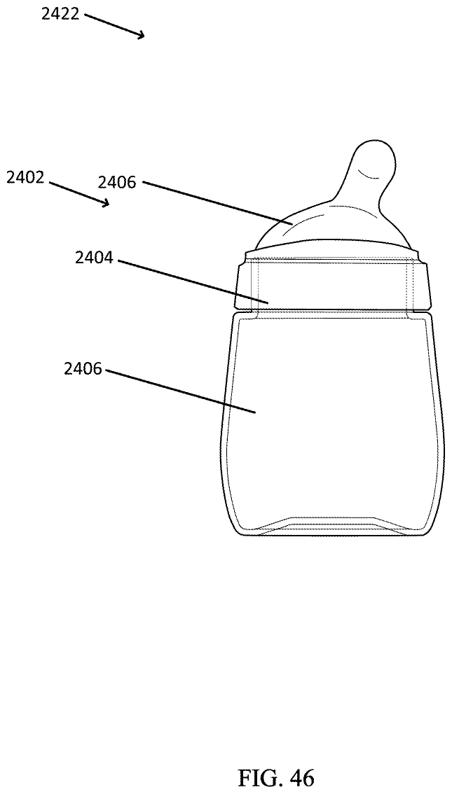

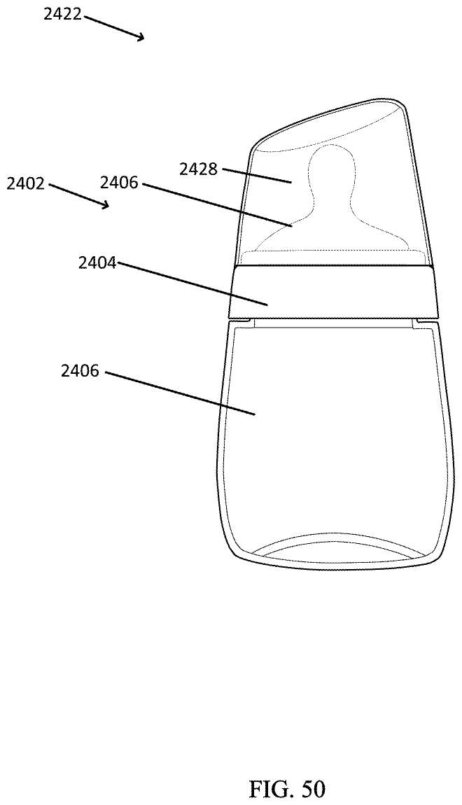

FIGS. 45-51 are front views of an example container 2422 with a nipple attachment 2402 used in place of a lid. The nipple attachment of FIGS. 45-51 may be used with the active attachment breastmilk storage system or passive breastmilk storage systems components. FIGS. 45-46 depict the nipple attachment 2402 positioned in different directions. In some examples, the lid of the container 2422 can be easily removed by a user and replaced by the nipple attachment 2402. The nipple attachment 2402 allows the same bottle 2406 to be used to store breastmilk and feed a baby. The nipple attachment 2402 includes a lid portion 2404 and a nipple portion 2406. The lid portion 2404 is secured to the bottle 2406 and prevents the breastmilk therein from being spilled while the nipple attachment 2402 is in use. In some examples, the lid portion 2404 funnels the breastmilk into the nipple portion 2406 when the container 2422 is tilted so that the bottle 2406 is positioned above the nipple attachment 2402. The nipple portion 2406 is attached to the lid portion 2404. In some examples, the nipple portion 2406 provides a surface for a baby to suck and extract breastmilk from the bottle 2406. In some examples, the nipple portion 2406 is flexible and can move between the various positions illustrated in FIGS. 45-46.

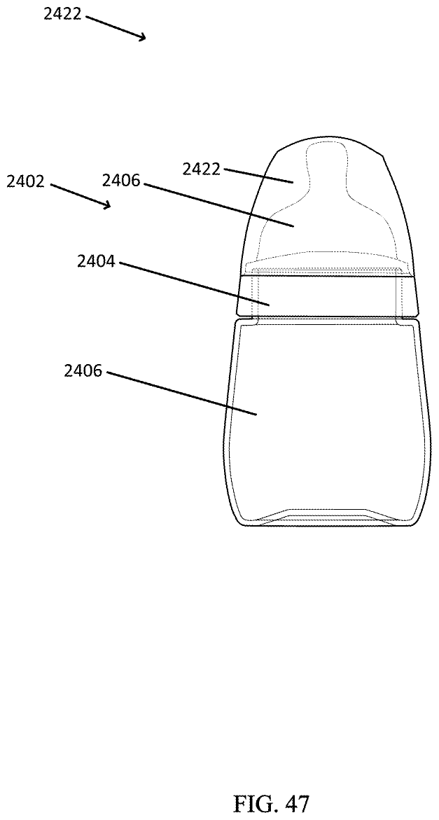

FIGS. 47-50 show the example container 2422 including various different covers. In the examples of FIGS. 47-50, the nipple attachment 2402 further includes nipple covers 2422, 2424, 2426, 2428 that are placed over the nipple portion 2406 to keep the nipple portion 2406 clean and free of debris. In some examples, the nipple covers 2408 also protect against breastmilk spills from the nipple portion 2406 of the nipple attachment 2402. As depicted in FIGS. 47-50, the nipple portion 2406 and nipple covers 2422, 2424, 2426, 2428 may be used in various shapes and sizes.

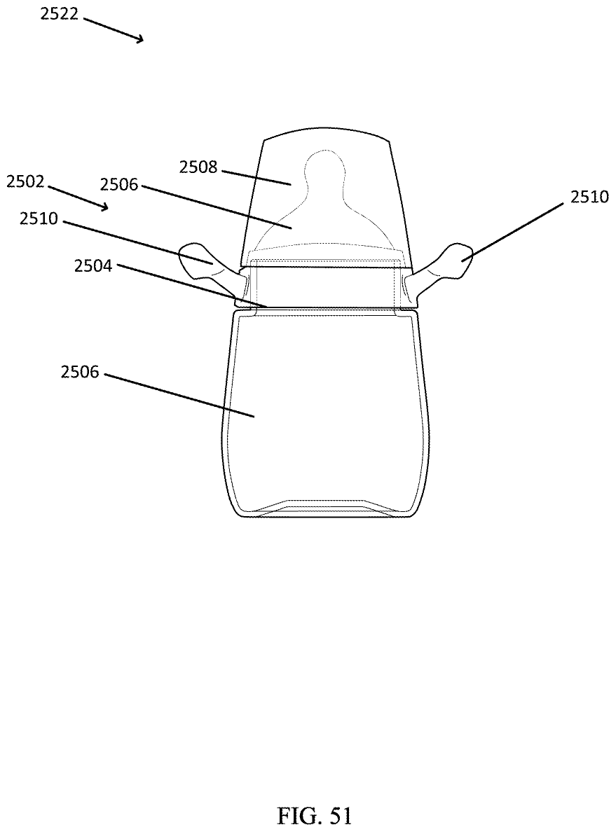

FIG. 51 is a front view of another example container 2522 with a nipple attachment 2502 used in place of a lid. In the example of FIG. 51, a lid portion 2504 further includes a handle 2510 on each side of the lid portion 2504. In some examples, the handles 2510 provide an easy place for the baby to grasp the container 2522 while feeding from the container 2522. In some examples, different numbers of handles 2510 may be positioned on the lid portion 2504. In other examples, handles 2510 may be alternatively placed on the side surface 2548 of the bottle 2506.

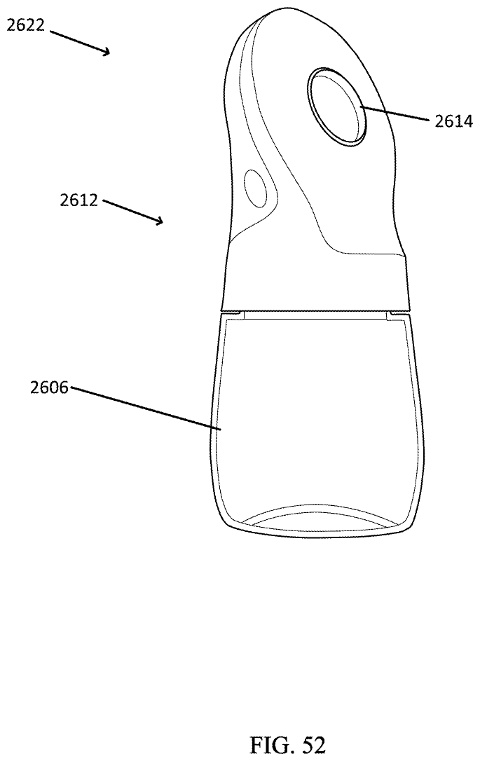

FIG. 52 is a front view of an example container 2622 with a breast contacting attachment 2612 used in place of a lid. In some examples the breast contacting attachment 2612 can be attached to the bottle 2606 so that a woman can easily fill the bottle 2606 with breastmilk. In this example, a woman may place an opening 2614 of the breast contacting attachment over her breast and express breastmilk into the opening 2614 of the breast contacting attachment 2612, thereby filling the bottle 2606. After the woman is finished expelling breastmilk into the bottle 2606, the breast contacting attachment 2612 can be removed and replaced with a nipple attachment 2602 or lid 2626.

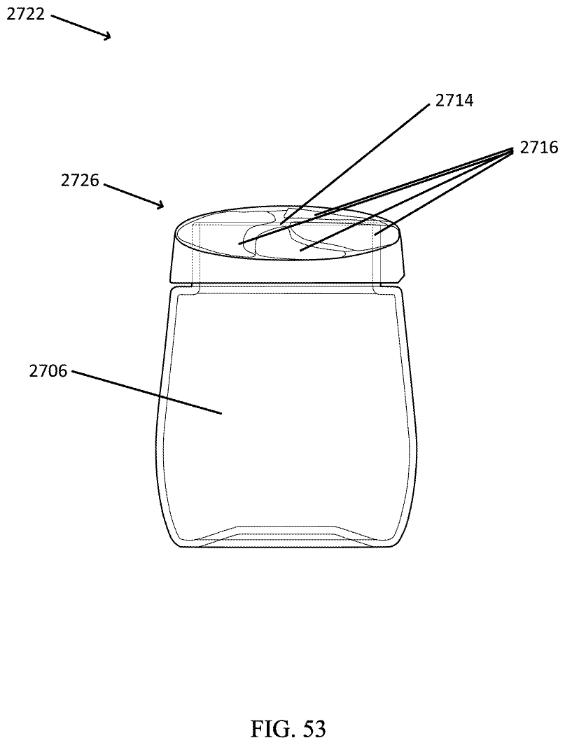

FIG. 53 is a front view on an example container 2722 with a penetrable lid 2726. In this example, the penetrable lid includes flaps 2716 and an opening 2714. In some examples, the flaps 2716 cover the opening 2714 of the lid. In some examples, the flips are made from a flexible plastic or rubber material and can be moved by a user or a baby to enlarge the opening 2714. In some examples, the container is filled with food. In these examples, a baby or user can reach through the opening while pushing the flaps away and access the food stored within the container. As the user withdraws the food from the interior of the container, the flaps flex back to their original position and cover the opening 2714 of the container 2722. In some examples, the flaps work together to prevent the contents of the container from being spilled if, for example, the container is knocked over or held upside down. In some examples, the container 2722 is able to be used with other containers 2722, and the containers may be stacked on top of each other, as explained in other embodiments herein. It should be recognized that in some examples, the containers 2722, function in accordance with principles of any of the passive or active attachment breastmilk storage systems described herein.

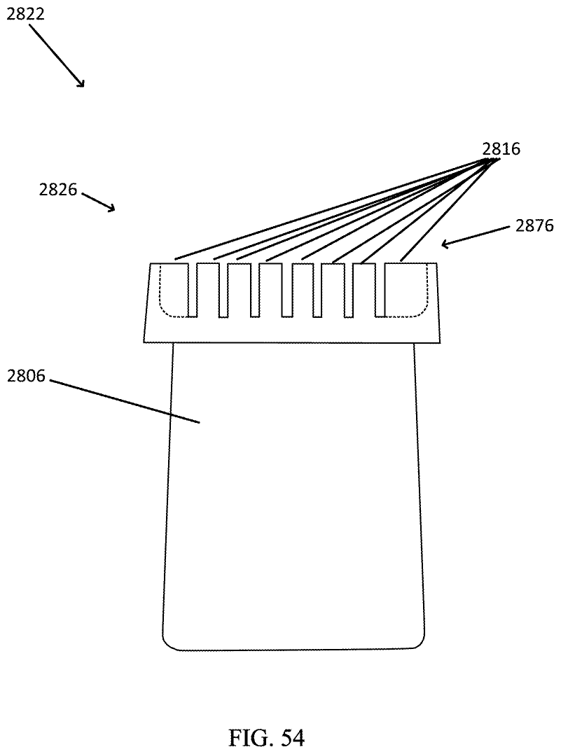

FIG. 54 is a front view of another example container 2822. As previously discussed with reference to FIGS. 26-28 and 30-31, in some examples, the container 2822 lids 2826 include a retaining flange 2876 that extends around the edge of the upper surface of the lid 2876. In such examples, the retaining flange 2876 may include gaps around its perimeter. In the example of FIG. 54, the retaining flange 2876 includes many gaps so that rather than appearing as a continuous flange, the retaining flange 2876 is made up of many structures 2816 positioned along the edge of the lid 2826. In some examples, the structures 2816 are flexible. In some examples, the flexible structures 2816 are each equal in size, while in other examples, the flexible structures 2816 are unequal in size so that some of the flexible structures 2816 extend further around the edge of the lid 2826 than others. The flexible structures 2816 may be made from various plastic, rubber, or metal materials such as, for example, engineering plastic, TPE, or silicone.

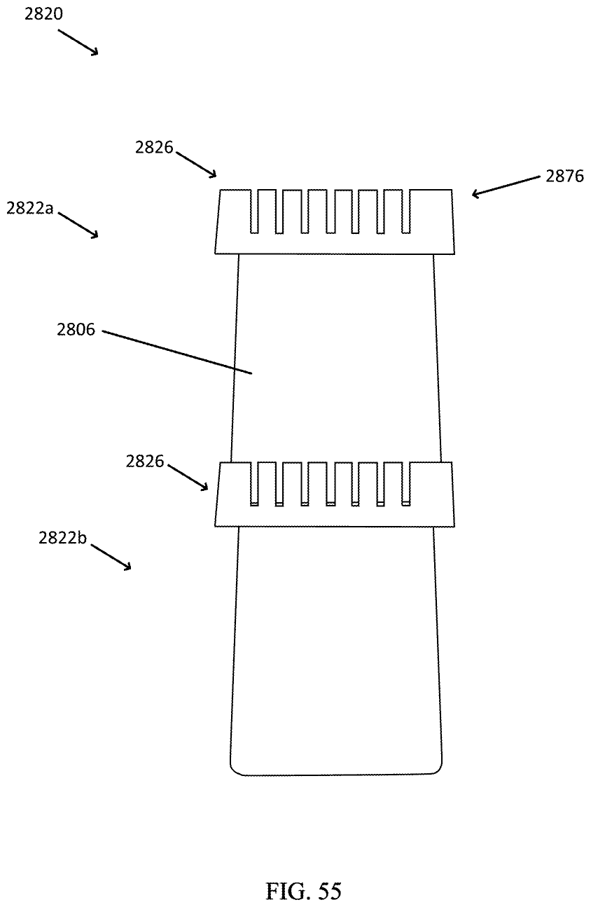

FIG. 55 is a front view of another example passive attachment breastmilk storage system 2820 utilizing the example containers 2822 of FIG. 53. As seen in the example of FIG. 55, the top container 2822a is placed on the bottom container 2822b so that the bottle 2806 of the top container 2822a rests on top of the lid 2826 of the bottom container 2822b. In the example of FIG. 55, the flexible structures 2816 are configured to flex and conform around the bottle 2806 of the top container 2822a as to help secure it in place in its stacked position. In this example, the flexible structures 2816 permit flexibility in the selection of bottle shapes, as the flexible structures 2816 are able to conform around various shaped bottles.

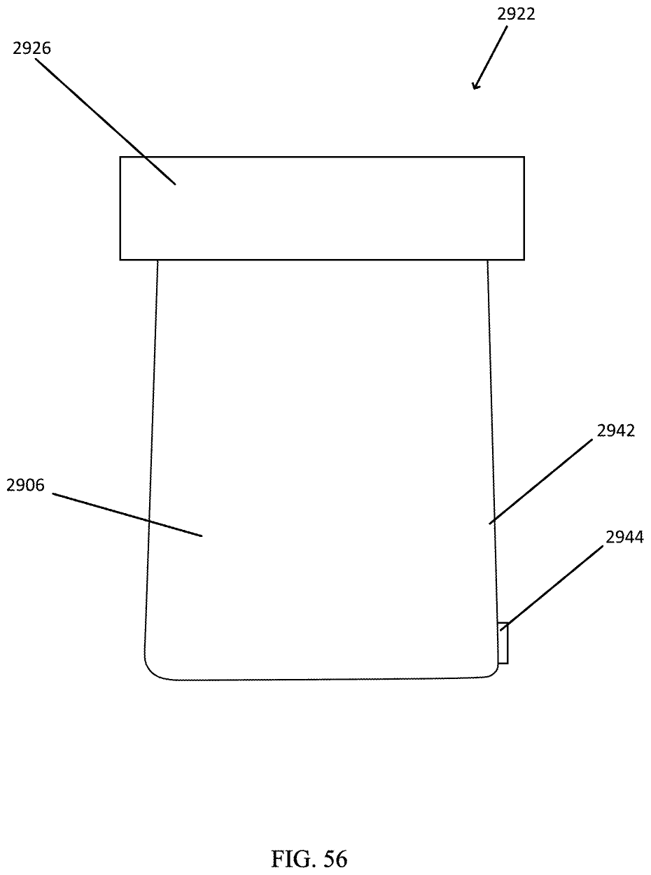

FIG. 56 is side view of another example container 2922. In the example of FIG. 56, the container 2922 includes a bottle 2906 and a lid 2926. The bottle 2906 further includes a protrusion 2944 extending out from the side surface 2942. In some examples, the protrusion 2944 is positioned near the bottom of the bottle 2906 on the side surface 2942. The protrusion 2944 may take various shapes. In some examples, the protrusion 2944 has a circular cross section, while in other examples, the protrusion 2944 is square or oval shaped. In some examples, protrusions are placed at several locations around the side surface 2942, while in other examples, such as the example of FIG. 55, only one protrusion 2944 is positioned on each bottle 2906.

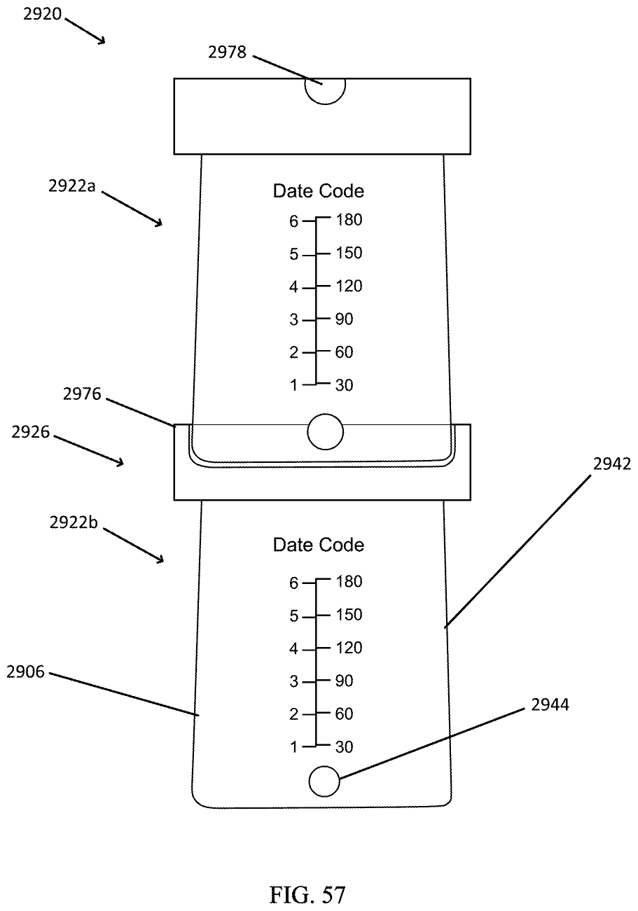

FIG. 57 is a front view of another example passive attachment breastmilk storage system 2920 using the containers 2922 of FIG. 56. As noted previously, the containers 2922 include a lid 2926 and a bottle 2906. In other examples, the containers 2922 are identical to the containers 2922 of FIG. 30, with the one difference being the inclusion of the protrusion 2944 on the side of the bottle 2906, as described with reference to FIG. 56.

In the example of FIG. 57, the top container 2922a is configured to be placed on top of the lid 2926 of the bottom container 2922b. The lid 2926 includes a retaining flange 2976 that extends around the edge of the lid 2926. In some examples, the retaining flange 2976 includes a cutout 2978. The cutout 2978 can be formed in various shapes. In some examples, the cutout 2978 is shaped complementary to the protrusion 2944 on the bottle 2906. In such examples, the bottle 2906 must be oriented on the lid 2926 so that the protrusion 2944 is aligned with the cutout 2978. Otherwise, the protrusion 2944 may interfere with the retaining flange 2976 and prevent the bottle 2906 of the top container 2922a from resting on the upper surface of the lid 2926 of the bottom container 2922b. Thus, when placing the top container 2922a on the bottom container 2922b, the user must rotate the top container 2922a until the protrusion 2944 and cutout 2978 line up.

In some examples, the lid 2926 is configured to be placed on the bottle 2906 of its respective container 2922 so that the cutout 2978 of the container 2922 is positioned vertically to and laterally in-line with the protrusion 2944 on the bottle 2906 of that same container 2922. In this case, the cutout 2978 of the top container 2922a would therefore also be vertical to and laterally in-line with the protrusion 2944 of the bottom container 2922b. In some examples, the bottle 2906 of the containers 2922 may include information on the side surface 2942, such as volumetric graduations, date information, or information about the contents of the containers 2922. If this information is located at the same position with respect to the protrusion 2944 on each bottle 2906 of the container 2922, it may be useful to employ the features of FIG. 57. By using the features of FIG. 57, the containers 2922 are rotatably fixed so that that the bottles 2906 are rotatably lined up with each other. Thus, the system can be utilized so that the information included on the side surfaces 2942 of each bottle 2906 is viewable from the same angle when placed in the stacked position.

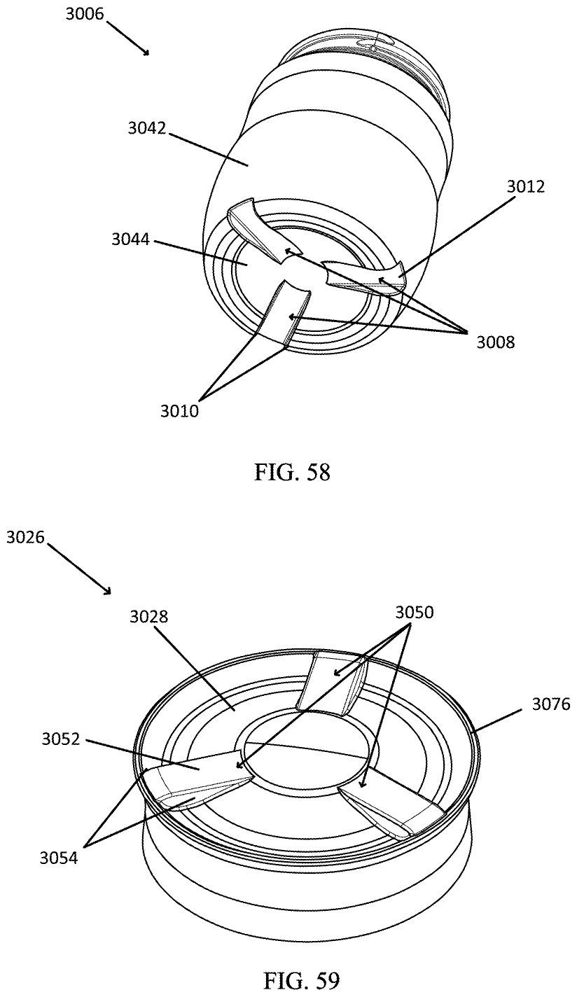

FIG. 58 is a bottom perspective view of another example bottle 3006. The example bottle 3006 includes a side surface 3042 and a bottom surface 3044. The bottle 3006 also includes a plurality of slots 3008 in the bottom surface 3044. In some examples, as in the example of FIG. 58, three slots 3008 are included on the bottom surface 3044. The slots 3008 each include a flat portion 3012 and two sidewall portions 3010. In the example of FIG. 58, the slots 3008 are placed equidistantly from each other around the bottom surface 3044 and extend from the edge of the bottom surface 3044 to the middle of the bottom surface 3044. In some examples, as in the example of FIG. 58, the slots 3008 are deepest at the edge of the bottom surface 3044 and gradually decrease in depth as they extend towards the middle of the bottom surface 3044. In some examples, the slots 3008 do not extend all the way to the middle of the bottom surface 3044.

FIG. 59 is a perspective view of an example lid 3026, configured to be used with the example bottle 3006 of FIG. 58. The example lid 3026 includes a retaining flange 3076 and an upper surface 3028. The lid 3026 also includes a plurality of ridges 3050. In some examples, as in the example of FIG. 59, three ridges 3050 are included on the upper surface 3028. The ridges 3050 and upper surface 3028 of the lid of FIG. 59 generally are shaped inverse to and complementary with the slots 3008 and bottom surface 3044 of the bottle 3006 of FIG. 58. Like the slots 3008 of the bottle 3006, the ridges 3050 vary in height, gradually getting shorter towards the middle of the upper surface 3028 of the lid 3026. In some examples, the ridges 3050 do not extend all the way to the middle of the upper surface 3028, so that the middle of the upper surface 3028 is a generally flat surface.

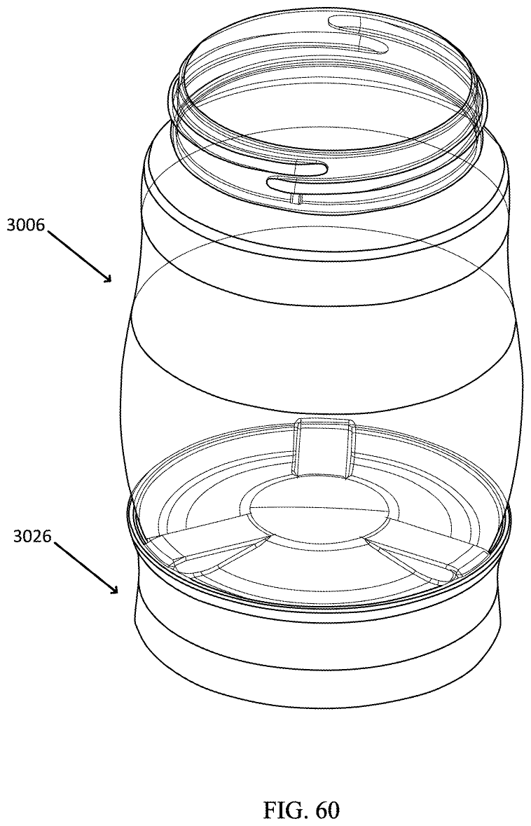

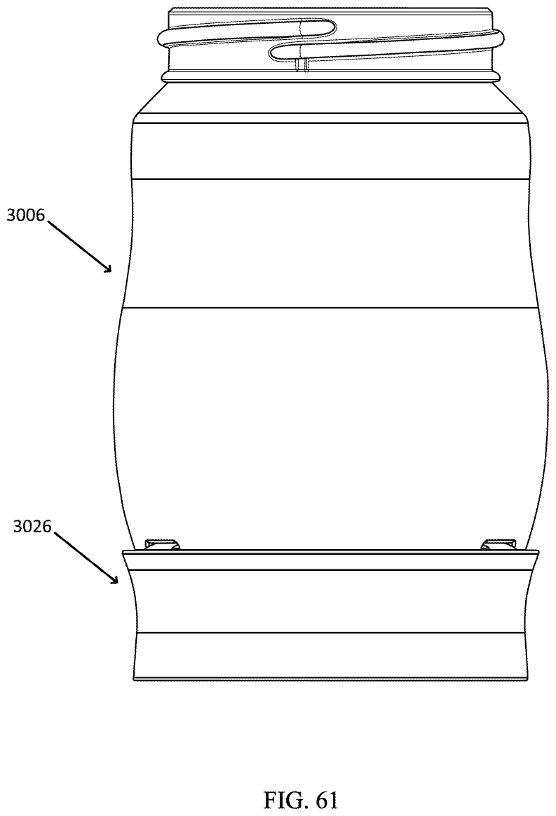

FIG. 60 is a semi-transparent perspective view of the example bottle 3006 of FIG. 58 stacked on top of the example lid 3026 of FIG. 59. FIG. 61 is a front view of the example bottle 3006 of FIG. 58 stacked on top of the example lid 3026 of FIG. 59. As depicted in FIG. 60 the bottom surface 3044 of the bottle 3006 is coincident to the upper surface 3028 of the lid 3026 when the bottle 3006 is placed on top of the lid 3026. Further, the bottle 3006 and lid 3026 are oriented so that the ridges 3050 of the lid 3026 are placed within the slots 3008 of the bottle 3006. In some embodiments, the ridges 3050 vary in shape from one another and the slots 3008 vary in shape from one another so that each ridge 3050 is shaped complementary to only one slot 3008. In this case, the bottle 3006 only is able to be stacked on the lid 3026 in a single radial orientation. In other examples, all of the ridges 3050 and slots 3008 are shaped the same, so that the bottle 3006 and lid 3026 may be stacked in in any radial orientation where the ridges 3050 and slots 3008 line up together. As depicted in FIG. 60, the lid 3026 and bottle 3006 each include three of the ridges 3050 or slots 3008. However, in other examples, fewer or greater ridges 3050 or slots 3008 can be used. In such cases, the bottle 3006 and lid 3026 can be stacked in fewer or greater radial orientations relative to each other. In some examples, once the ridges 3050 and slots 3008 are oriented in alignment with each other, a user can press down on the bottle 3006 to initiate a press fit between the bottle 3006 and the lid 3026. Thus, when the bottle 3006 is pressed into the ridges 3050 of the lid 3026, the ridges 3050 and slots 3008 fit together. The alignment of the ridges 3050 and slots 3008 prohibit rotational movement of the bottle 3006 and the lid 3026 relative to each other while the bottle 3006 is stacked on top of the lid 3026. In some examples, the press fit initiated between the lid 3026 and the bottle 3006 is releasable by a user by pulling up on the bottle 3006 while pulling down on the lid 3026. Although in the example of FIG. 60, a bottle 3006 is shown without a lid on top of it and a lid 3026 is shown without a bottle underneath of it, it should be appreciated that another lid, as depicted in FIG. 59, could be placed on top of the bottle 3006 while the bottle 3006 bottom surface is on top of the lid 3026. Likewise, another bottle, such as the one depicted in FIG. 58, could be included under the lid 3026 shown in the example of FIG. 60 while the lid 3026 is placed under the stacked bottle 3006.

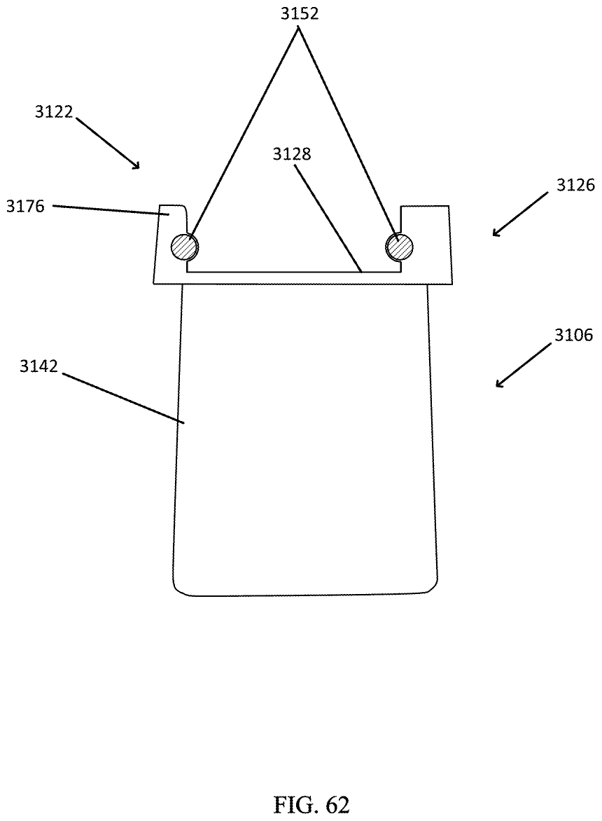

FIG. 62 is a front view of another example container 3122. In the example of FIG. 62, the container 3122 includes a bottle 3106 and a lid 3126. In some examples, as depicted in FIG. 62, the side surfaces 3142 of the bottle 3106 are straight and smoother. In other examples, the side surfaces 3142 include a lid attachment 3128 feature adjacent to the bottom edge of the bottle 3106, as previously described with reference to FIG. 39.

In some examples, the lid 3126 of the container 3122 includes an upper surface 3128 and a retaining flange 3176. In some examples, the upper surface 3128 is flat, while in other examples, the upper surface 3128 is contoured and curved. In some examples, the upper surface 3128 is shaped complementary to a bottom surface of a bottle 3106. In some examples, the retaining flange 3176 also includes bottle attachment features 3152, as described with reference to the example of FIG. 39. However, in contrast to the example of FIG. 39, where the bottle attachment features are positioned directly on the upper edge of the retaining flange, in the example of FIG. 62, the bottle attachment features 3152 are positioned below an upper edge of the retaining flange 3176.

In some examples, the bottle attachment features 3152 are formed from a flexible material, capable of deforming when manipulated. In some examples, the flexible material is engineering plastic, TPE, or silicone.

In some examples, as with other embodiments described herein, the retaining flange 3176 and bottle attachment features 3152 extend around the entire edge of the lid 3126. Likewise, in other examples such as the example of FIG. 62, the retaining flange 3176 and bottle attachment features 3152 only extend partway around the edge of the lid 3126 so that there are multiple distinct sections of the retaining flange 3176 and bottle attachment features 3152. Thus, in all examples described herein, it should be recognized that although “retaining flange” is referred to in its singular form, the retaining flange may consist of multiple flange portions, rather than a continuous singular flange.

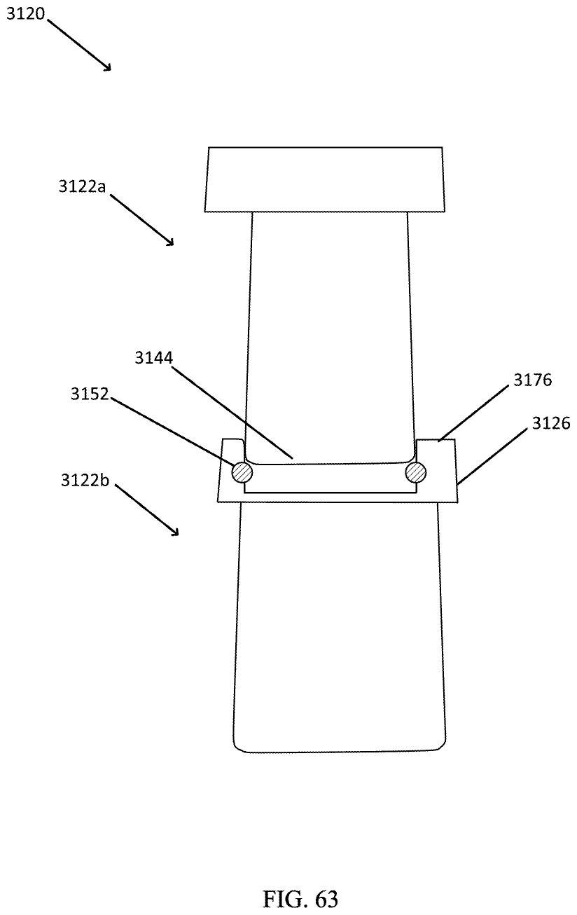

FIG. 63 is a front view of a hybrid attachment breastmilk storage system 3120 using the example container 3122 of FIG. 62. In the example of FIG. 63, a top container 3122a is placed on top of a bottom container 3122b. The top container 3122a bottle 3106 is positioned to sit within the retaining flange 3176 of the lid 3126 of the bottom container 3122b. In the example of FIG. 63, the bottle 3106 sits on top of, but does not engage with, the bottle retaining features 3152. Rather, the bottle attachment features 3152 simply support the edge of the bottom surface 3144 of the bottle 3106. In this example, the retaining flange 3176 provides some support to prevent the top container 3122a from being easily knocked off its position on top of the lid 3126 of the bottom container 3122b. In some examples, as shown in FIG. 63, the two containers 3122 have different style lids. In other examples, the lid of the top container and the bottom container are the same style.

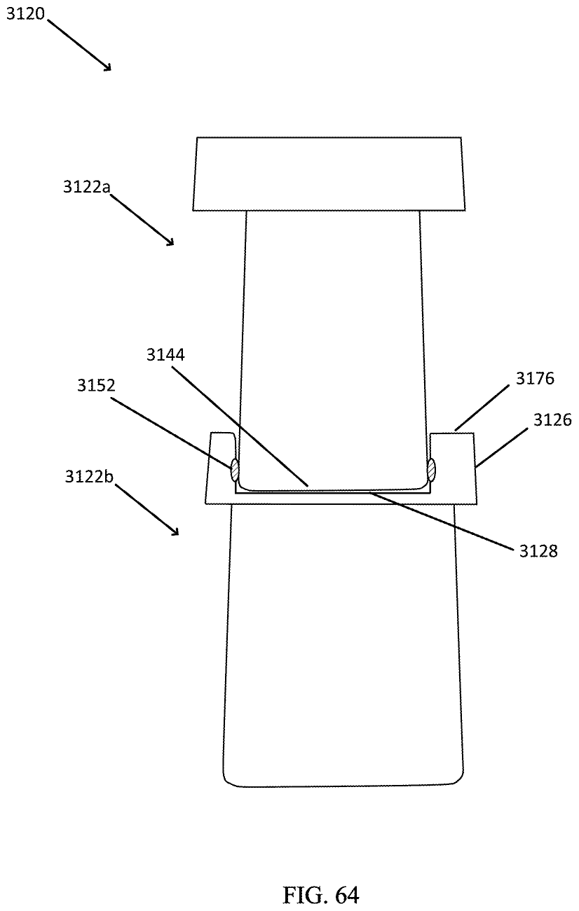

FIG. 64 is another front view of the hybrid attachment breastmilk storage system 3120 of FIG. 63. In the example of FIG. 64, the top container 3122a is positioned within the retaining flange 3176 and is supported by upper surface 3128 of the lid 3126 of the bottom container 3122b. When the top container 3122a is fully seated within the retaining flange 3176, as depicted in FIG. 64, the bottle retaining features 3152 conform around the inserted bottle 3106 to secure it in place. The fit provided by the retaining flange 3176 and bottle attachment features 3152 prevents the top container 3122a from easily being knocked out of its position on top of the bottom container 3122b. In some examples, as shown in FIG. 64, the bottle attachment features 3152 do not mate with any corresponding lid attachment features on the bottle. However, in other examples, the bottle 3106 may be provided with one or more lid attachment features provided thereon. In these examples, the lid attachment features are configured to mate with the flexible bottle attachment features of the lid.

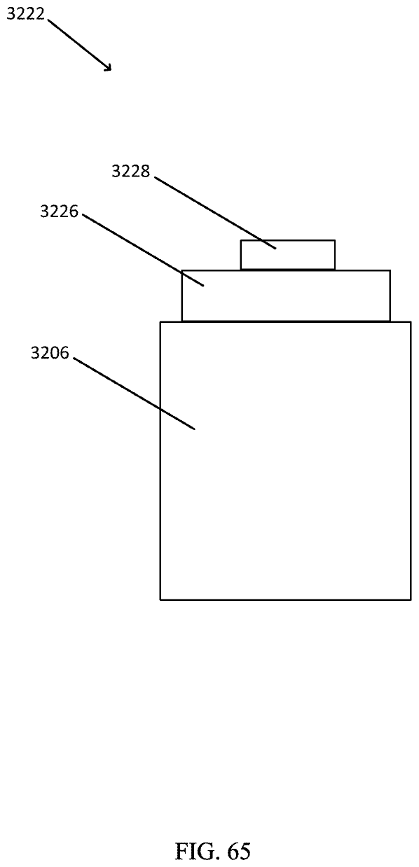

FIG. 65 is a front view of another example container 3222. In the example of FIG. 65, the container includes a lid 3226 and a bottle 3206. The lid 3226 and the bottle 3206 both include threads for securing the lid 3226 and the bottle 3206 to each other and sealing the interior space of the bottle 3206. The lid 3226 also includes a stacking feature 3228 for stacking a bottle of another container on top of the lid 3226. Specifically, the stacking feature 3228 extends from the lid 3226 and engages a notch on the bottom of another container placed on top of the container 3222 shown to couple the containers.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A breastmilk storage system comprising:
a first bottle with a first associated lid;
wherein the first bottle is configured to be stacked on top of a second associated lid of a second bottle while the first associated lid is covering an opening of the first bottle.

2. The breastmilk storage system of claim 1, wherein the top surface of the first associated lid is configured to mate with a bottom surface of the second bottle.

3. The breastmilk storage system of claim 2, wherein the top surface of the first associated lid has a convex shape.

4. The breastmilk storage system of claim 3, wherein the top surface of the first associated lid is shaped complementary to the bottom surface of the second bottle.

5. The breastmilk storage system of claim 2, wherein the first associated lid includes an upwardly extending flange around an edge of the first associated lid.

6. The breastmilk storage system of claim 5, wherein the upwardly extending flange is continuous around an entire perimeter of the first associated lid.

7. The breastmilk storage system of claim 5, wherein the upwardly extending flange includes attachment features that are configured to engage with a ridge of the second bottle and secure the second bottle to the first associated lid.

8. The breastmilk storage system of claim 7, wherein the ridge and the attachment features are mating threads.

9. The breastmilk storage system of claim 2, wherein the top surface of the first associated lid includes an upwardly extending protrusion.

10. The breastmilk storage system of claim 9, wherein the upwardly extending protrusion is shaped complementary to a recess in the bottom surface of the second bottle.

11. The breastmilk storage system of claim 10, wherein the upwardly extending protrusion is configured to mate with the recess when the second bottle is placed on top of the first associated lid.

12. The breastmilk storage system of claim 11, wherein the upwardly extending protrusion and the recess include attachment features that engage with each other and secure the second bottle to the first associated lid.

13. A method for stacking breastmilk storage containers, the method comprising:
providing a lid and a bottle; and
positioning the bottle on top of the lid.

14. The method of claim 13, wherein the lid is positioned over an opening of another bottle.

15. The method of claim 13, wherein the lid is shaped complementary to a bottom surface of the bottle.

16. The method of claim 15, wherein the lid and the bottom surface of the bottle include attachment features that secure the bottle to the lid.

17. A breastmilk storage system comprising:
a first bottle with a first associated lid; and
a second bottle with a second associated lid;
wherein the first bottle includes a bottom that is configured to mate with and stack on top of the second associated lid; and
wherein the second bottle includes a bottom that is configured to mate with and stack on top of the first associated lid.

18. The breastmilk storage system of claim 17, wherein the bottom of the second bottle and the bottom of the first bottle each include a recess therein.

19. The breastmilk storage system of claim 18, wherein:
the recess of the bottom of the first bottle is configured to receive a portion of the second associated lid; and
the recess of the bottom of the second bottle is configured to receive a portion of the first associated lid.

20. The breastmilk storage system of claim 19, wherein
the first associated lid includes a flange configured to wrap around a portion of the bottom of the second bottle; and
wherein the second associated lid includes a flange configured to wrap around the portion of the bottom of the first bottle.
